SEQUENCE LISTING (9 sequences)

SEQ ID NO: 1 - DNA, 1821 bp, Bacillus subtilis
gtggaacaggatacgcagcatgttaaaccacttcaaacaaaaaccgatattcatgcagtcttggcctctaatggacgcatcatttatatatctgccaactccaaactgcatttgggctatctccaaggagagatgatcggatcattcctcaaaacgtttctgcatgaggaagaccaatttttggttgaaagctatttttataatgaacatcatctgatgccgtgcacctttcgttttattaaaaaagatcatacgattgtgtgggtggaggctgcggtagaaattgttacgacaagagctgagcggacagaacgggaaatcattttgaaaatgaaggttcttgaagaagaaacaggccatcaatccctaaactgcgaaaaacatgaaatcgaacctgcaagcccggaatcgactacatatataacggatgattatgaacggttggttgaaaatctcccgagtccgctatgcatcagtgtcaaaggcaagatcgtctatgtaaacagcgcgatgctttcaatgctgggagccaaaagcaaggatgctattattggtaaatcgtcctatgaatttattgaagaagaatatcatgatatcgtgaaaaacaggattatacgaatgcaaaaaggaatggaagtcggaatgattgaacagacgtggaaaaggcttgatggcacacctgttcatttagaagtgaaagcatccccgaccgtctacaaaaaccagcaggctgagctgctgctgctgatcgatatctcttcaaggaaaaaattccaaaccatcctgcaaaaaagccgtgaacgatatcagctgctgattcaaaattccattgataccattgcggtgattcacaatggaaaatgggtatttatgaatgaatcgggaatttccctgtttgaagcggctacatatgaggatttaattggcaaaaacatatacgatcagctgcatccttgcgatcacgaggatgtaaaagagagaatccaaaacattgccgagcaaaaaacagaatctgaaattgtcaagcaatcctggttcacctttcagaacagggtcatctatacggagatggtctgcattccgacgaccttttttggtgaagcggccgtccaggtcattcttcgggacatctcagagagaaaacaaacagaagaattgatgctgaaatcggaaaaattatcaatcgcagggcagctcgcggcgggaatcgcccatgagatccgcaaccctcttacagcgatcaaaggatttttacagctgatgaaaccgacaatggaaggcaacgaacattactttgatattgtgttttctgaactcagccgtatcgaattaatactcagtgaactgctcatgctggcgaaacctcagcaaaatgctgtcaaagaatatttgaacttgaaaaaattaattggtgaggtttcagccctgttagaaacgcaggcgaatttaaatggcatttttatcagaacaagttatgaaaaagacagcatttatataaacggggatcaaaaccaattaaagcaggtattcattaatttaatcaaaaatgcagttgaatcaatgcctgatgggggaacagtagacattatcataaccgaagatgagcattctgttcatgttactgtcaaagacgaaggggaaggtatacctgaaaaggtactaaaccggattggagagccatttttaacaacaaaagaaaaaggtacggggcttggattaatggtgacatttaatatcattgaaaaccatcagggagttatacatgtggacagccatcctgaaaaaggcacagcgtttaaaatttcatttccaaaaaaataa

SEQ ID NO: 2 - protein, 606 aa, unknown
  Bacillus sp.
MEQDTQHVKPLQTKTDIHAVLASNGRIIYISANSKLHLGYLQGEMIGSFLKTFLHEEDQFLVESYFYNEHHLMPCTFRFIKKDHTIVWVEAAVEIVTTRAERTEREIILKMKVLEEETGHQSLNCEKHEIEPASPESTTYITDDYERLVENLPSPLCISVKGKIVYVNSAMLSMLGAKSKDAIIGKSSYEFIEEEYHDIVKNRIIRMQKGMEVGMIEQTWKRLDGTPVHLEVKASPTVYKNQQAELLLLIDISSRKKFQTILQKSRERYQLLIQNSIDTIAVIHNGKWVFMNESGISLFEAATYEDLIGKNIYDQLHPCDHEDVKERIQNIAEQKTESEIVKQSWFTFQNRVIYTEMVCIPTTFFGEAAVQVILRDISERKQTEELMLKSEKLSIAGQLAAGIAHEIRNPLTAIKGFLQLMKPTMEGNEHYFDIVFSELSRIELILSELLMLAKPQQNAVKEYLNLKKLIGEVSALLETQANLNGIFIRTSYEKDSIYINGDQNQLKQVFINLIKNAVESMPDGGTVDIIITEDEHSVHVTVKDEGEGIPEKVLNRIGEPFLTTKEKGTGLGLMVTFNIIENHQGVIHVDSHPEKGTAFKISFPKK

SEQ ID NO: 3 - protein, 425 aa, Unknown
  Bacillus sp.
LTAPSIKSGTILHAWNWSFNTLKHNMKDIHDAGYTAIQTSPINQVKEGNQGDKSMSNWYWLYQPTSYQIGNRYLGTEQEFKEMCAAAEEYGIKVIVDAVINHTTSDYAAISNEVKSIPNWTHGNTQIKNWSDRWDVTQNSLLGLYDWNTQNTQVQSYLKRFLDRALNDGADGFRFDAAKHIELPDDGSYGSQFWPNITNTSAEFQYGEILQDSASRDAAYANYMDVTASNYGHSIRSALKNRNLGVSNISHYASDVSADKLVTWVESHDTYANDDEESTWMSDDDIRLGWAVIASRSGSTPLFFSRPEGGGNGVRFPGKSQIGDRGSALFEDQAITAVNRFHNVMAGQPEELSNPNGNNQIFMNQRGSHGVVLANAGSSSVSINTATKLPDGRYDNKAGAGSFQVNDGKLTGTINARSVAVLYPD

SEQ ID NO: 4 - protein, 275 aa, Bacillus amyloliquefaciens
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 5 - protein, 238 aa, Ptilosarcus gurneyi
VNRNVLKNTGLKEIMSAKASVEGIVNNHVFSMEGFGKGNVLFGNQLMQIRVTKGGPLPFAFDIVSIAFQYGNRTFTKYPDDIADYFVQSFPAGFFYERNLRFEDGAIVDIRSDISLEDDKFHYKVEYRGNGFPSNGPVMQKAILGMEPSFEVVYMNSGVLVGEVDLVYKLESGNYYSCHMKTFYRSKGGVKEFPEYHFIHHRLEKTYVEEGSFVEQHETAIAQLTTIGKPLGSLHEWV

SEQ ID NO: 6 - DNA, 135 bp, Bacillus subtilis
atgaaatctaaatggatgtcaggtttgttgctcgttgcggtcgggttcagctttactcaggtgatggttcatgcaggtgaaacagcaaacacagaagggaaaacatttcatattgcggcacgcaatcaaacatga

SEQ ID NO: 7 - protein, 44 aa, unknown
  Bacillus sp.
MKSKWMSGLLLVAVGFSFTQVMVHAGETANTEGKTFHIAARNQT

SEQ ID NO: 8 - DNA, 135 bp, Bacillus subtilis
atgaaatctaaattgtttatcagtttatccgccgttttaattggacttgcctttttcggatctatgtataatggcgaaatgaaggaagcatcccggaatgtaactctcgcacctactcatgaattccttgtttaa

SEQ ID NO: 9 - protein, 44 aa, unknown
  Bacillus sp.
MKSKLFISLSAVLIGLAFFGSMYNGEMKEASRNVTLAPTHEFLV

(19) United States
(12) Patent Application Publication
(10) Pub. No.: US 2017/0369537 A1
(43) Pub. Date: Dec. 28, 2017

(54) ENHANCED PROTEIN EXPRESSION

(71) Applicant: DANISCO US INC., Palo Alto, CA (US)

(72) Inventors: Cristina BONGIORNI, Fremont, CA (US); Robert I. CHRISTENSEN, Pinole, CA (US); Brian F. SCHMIDT, Half Moon Bay, CA (US); Anita VAN KIMMENADE, Woodside, CA (US)

(73) Assignee: DANISCO US INC., Palo Alto, CA (US)

(21) Appl. No.: 15/533,487

(22) PCT Filed: Dec. 11, 2015
(86) PCT No.: PCT/US2015/065296

§ 371 Date: Jun. 6, 2017

Related U.S. Application Data
(60) Provisional application No. 62/094,751, filed on Dec. 19, 2014.

(51) Int. Cl. C07K 14/32; C12N 9/54; C12N 9/88; C12N 15/75; C12N 9/12; C12R 1/125; C12P 21/02

(52) U.S. Cl. CPC C07K 14/32; C12N 15/75; C12N 9/54; C12N 9/12; C12N 9/88; C12P 21/02; C12R 1/125

(57) ABSTRACT

The present invention relates in general to bacterial cells having a genetic alteration that results in increased expression of a protein of interest and methods of making and using such cells. Aspects of the present invention include Grampositive microorganisms, such as members of the Bacillus genus having a genetic alteration that delays, reduces, or blocks the expression or activation of genes for sporulation, thereby resulting in enhanced expression of a protein of interest. The genetic alteration is one that reduces expression of a kinA gene, a phrA gene or a phrE gene.

### CROSS-REFERENCE TO RELATED APPLICATIONS

This application claims priority under 35 U.S.C. §119 to U.S. Provisional Patent Application No. 62/094,751, filed Dec. 19, 2014, the entirety of which is hereby incorporated by reference.

### FIELD OF THE INVENTION

The present invention relates in general to bacterial cells having a genetic alteration that results in increased expression of a protein of interest and methods of making and using such cells. Aspects of the present invention include Gram-positive microorganisms, such as members of the Bacillus genus, having a genetic alteration that delays, reduces, or blocks the expression or activation of genes for sporulation, thereby resulting in enhanced expression of a protein of interest. Examples of genetic alterations include those that reduce the expression or activity of KinA, PhrA, and/or PhrE.

### REFERENCE TO SEQUENCE LISTING

The contents of the electronic submission of the text file Sequence Listing, which is named NB40522-WO-PCT_SequenceListing.txt, was created on Nov. 30, 2015 and is 18 KB in size, is incorporated herein by reference.

### BACKGROUND OF THE INVENTION

Genetic engineering has allowed the improvement of microorganisms used as industrial bioreactors, cell factories and in food fermentations. Gram-positive organisms, including a number of Bacillus species, are used to produce a large number of useful proteins and metabolites (see, e.g., Zukowski, “Production of commercially valuable products,” In: Doi and McGlouglin (eds.) Biology of Bacilli: Applications to Industry, Butterworth-Heinemann, Stoneham. Mass. pp 311-337 [1992]). Common Bacillus species used in industry include B. licheniformis, B. amyloliquefaciens and B. subtilis. Because of their GRAS (generally recognized as safe) status, strains of these Bacillus species are natural candidates for the production of proteins utilized in the food and pharmaceutical industries. Examples of proteins produced in Gram-positive organisms include enzymes, e.g., α-amylases, neutral proteases, and alkaline (or serine) proteases.

In spite of advances in the understanding of production of proteins in bacterial host cells, there remains a need for to develop new recombinant strains that express increased levels of a protein of interest.

### SUMMARY OF THE INVENTION

The present invention provides recombinant Gram positive cells that express increased levels of a protein of interest and methods of making and using the same. In particular, the present invention relates to bacterial cells having a genetic alteration that results in increased expression of a protein of interest as compared to bacterial cells that do not have the genetic alteration. Aspects of the present invention therefore include Gram positive microorganisms, such as members of the genus Bacillus, comprising a genetic alteration that reduces the expression of a gene that functions to activate the phosphorelay pathway. (e.g., see phosphorelay pathway schematic in FIG. 5) and thus results in enhanced expression of a protein of interest (hereinafter, a “POI”). Methods of making and using such recombinant bacterial cells are also provided.

Aspects of the invention include methods for increasing expression of a POI from a Gram positive bacterial cell comprising (a) obtaining an altered Gram positive bacterial cell producing a POI, wherein the altered Gram positive bacterial cell comprises at least one genetic alteration that reduces expression or activity of one or more proteins that activate the phosphorelay pathway and (b) culturing said altered Gram positive bacterial cell under conditions such that the POI is expressed, wherein the increased expression of the POI is relative to the expression of the same POI in an unaltered (parental) Gram positive bacterial cell grown under essentially the same culture conditions. In certain embodiments, a genetic alteration that reduces the expression or activity of one or more proteins that activate the phosphorelay pathway is a genetic alteration of a kinA gene, a phrA gene and/or a phrE gene.

In certain other embodiments, the altered Gram positive cell is derived from a parental cell that has one or more defective or inactive sporulation genes (e.g., the genes whose expression is controlled by Spo0A or are downstream of Spo0A), and is thus already prevented from forming spores. For example, Applicant has observed that even in this non-sporulating genetic background, additional genetic alterations that reduce expression or activity of one or more proteins activating the phosphorelay pathway (i.e., genes that control the expression of sporulation-initiating genes) increase the expression of a POI from the cell. Therefore, the improvement in protein expression/production in the genetically altered (daughter) cells of the disclosure are not due solely to preventing sporulation of the Gram positive cell. For example, the parental Gram positive cells from which the altered Gram positive (daughter) cells of the disclosure are derived, can have a non-functional sporulation gene, a mutated sporulation gene, a deleted sporulation gene, and the like (e.g., see Examples section, which employ sporulation deficient Bacillus cells).

In certain embodiments, the altered Gram positive bacterial cell is a member of the Bacillus genus (e.g., Bacillus cells selected from the group consisting of B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii and B. thuringiensis. In certain embodiments, the Bacillus cell is a B. subtilis cell. In certain embodiments, the altered Gram positive bacterial cell further comprises a mutation in a gene selected from the group consisting of degU, degQ, degS, scoC4, and the like. In certain embodiments, the mutation is degU(Hy)32.

In certain embodiments, the genetic alteration results in a decrease in the level of expression of one or more of the kinA, phrA, and phrE genes in the altered Gram positive (daughter) bacterial cell as compared to a corresponding unaltered Gram positive (parental) bacterial cell grown under essentially the same culture conditions. Thus, the genetic alteration can results in a decrease in the level of expression of any one of the kinA, phrA, and phrE genes; any two of the kinA, phrA, and phrE genes; or all three of the kinA, phrA, and phrE genes. In other embodiments, the genetic alteration results in a decrease in the activity of one or more of the KinA, PhrA, and PhrE proteins in the altered Gram positive bacterial cell as compared to a corresponding unaltered Gram positive bacterial cell grown under essentially the same culture conditions. Thus, the genetic alteration can result in a decrease in the activity of any one of the KinA, PhrA, and PhrE proteins; any two of the KinA, PhrA, and PhrE proteins; or all three of the KinA, PhrA, and PhrE proteins.

In certain embodiments, the sequence of the wild type kinA gene is at least 60% identical to SEQ ID NO: 1, the sequence of the wild type phrA gene is at least 60% identical to SEQ ID NO: 6, and the sequence of the wild type phrE gene is at least 60% identical to SEQ ID NO: 8. In certain embodiments, the sequence of the wild type KinA protein is at least 80% identical to SEQ ID NO: 2, the sequence of the wild type PhrA protein is at least 80% identical to SEQ ID NO:7, and the sequence of the wild type PhrE protein is at least 80% identical to SEQ ID NO:9. In certain embodiments, the genetic alteration is a deletion of all or part of one or more of the kinA, phrA, and phrE genes.

In certain embodiments, the POI is a homologous protein. In certain embodiments, the POI is a heterologous protein. In certain embodiments, the POI is an enzyme. In certain embodiments, the enzyme is selected from the group consisting of protease, cellulase, pullulanase, amylase, carbohydrase, lipase, isomerase, transferase, kinase, and phosphatase. In certain other embodiments, the enzyme is selected from the group consisting of acetyl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, rhamno-galacturonases, ribonucleases, transferases, transport proteins, transglutaminases, xylanases, hexose oxidases, and combinations thereof.

In certain other embodiments, the POI is a protease. In certain embodiments, the protease is a subtilisin. In certain other embodiments, the subtilisin is selected from the group consisting of subtilisin 168, subtilisin BPN', subtilisin Carlsberg, subtilisin DY, subtilisin 147, subtilisin 309, and variants thereof.

In certain embodiments, the method further comprisies isolating and recovering the POI. In yet other embodiments the isolated and recovered POI is further purified.

Aspects of the present invention include an altered Gram positive bacterial cell, wherein said altered Gram positive bacterial cell comprises at least one genetic alteration that reduces the expression or activity of one or more proteins that activate the phosphorelay pathway that induces the expression of sporulation-initiating genes as compared to a corresponding unaltered Gram positive bacterial cell grown under essentially the same culture conditions. In certain embodiments, the altered Gram positive bacterial cell is a member of the Bacillus genus. In certain embodiments, the Bacillus cell is selected from the group consisting of B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii and B. thuringiensis. In certain other embodiments, the Bacillus cell is a B. subtilis cell. In certain embodiments, the altered Gram positive bacterial cell further comprises a mutation in a gene selected from the group consisting of degU, degQ, degS, scoC4 and the like. In certain embodiments, the mutation is degU(Hy)32.

In certain embodiments, the genetic alteration results in a decrease in the level of expression of one or more of the kinA, phrA, and phrE genes in the altered Gram positive bacterial cell as compared to a corresponding unaltered Gram positive bacterial cell grown under essentially the same culture conditions. Thus, the genetic alteration can results in a decrease in the level of expression of any one of the kinA, phrA, and phrE genes; any two of the kinA, phrA, and phrE genes; or all three of the kinA, phrA, and phrE genes. In other embodiments, the genetic alteration results in a decrease in the activity of one or more of the KinA, PhrA, and PhrE proteins in the altered Gram positive bacterial cell as compared to a corresponding unaltered Gram positive bacterial cell grown under essentially the same culture conditions. Thus, the genetic alteration can results in a decrease in the activity of: any one of the KinA, PhrA, and PhrE proteins; any two of the KinA, PhrA, and PhrE proteins; or all three of the KinA, PhrA, and PhrE proteins.

In certain embodiments, the sequence of the wild type kinA gene is at least 60% identical to SEQ ID NO:1, the sequence of the wild type phrA gene is at least 60% identical to SEQ ID NO:6, and the sequence of the wild type phrE gene is at least 60% identical to SEQ ID NO:8. In certain embodiments, the sequence of the wild type KinA protein is at least 80% identical to SEQ ID NO:2, the sequence of the wild type PhrA protein is at least 80% identical to SEQ ID NO:7, and the sequence of the wild type PhrE protein is at least 80% identical to SEQ ID NO:9. In certain embodiments, the genetic alteration is a deletion of all or part of one or more of the kinA, phrA, and phrE genes.

In certain embodiments, the altered cell expresses aPOI. In certain embodiments, the POI is a homologous protein. In certain embodiments, the POI is a heterologous protein. In certain embodiments, the POI is an enzyme.

In certain embodiments, the enzyme is selected from the group consisting of protease, cellulase, pullulanase, amylase, carbohydrase, lipase, isomerase, transferase, kinase, and phosphatase. In certain other embodiments, the enzyme is selected from the group consisting of acetyl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, rhamno-galacturonases, ribonucleases, transferases, transport proteins, transglutaminases, xylanases, hexose oxidases, and combinations thereof. In other embodiments, the POI is a protease. In certain embodiments, the protease is a subtilisin. In certain embodiments, the subtilisin is selected from the group consisting of: subtilisin 168, subtilisin BPN', subtilisin Carlsberg, subtilisin DY, subtilisin 147, subtilisin 309, and variants thereof.

In certain embodiments, the genetic alteration results in a decrease in the level of expression of one or more of the kinA, phrA, and phrE genes in the altered Gram positive bacterial cell as compared to a corresponding unaltered Gram positive bacterial cell grown under essentially the same culture conditions. Thus, the genetic alteration can results in a decrease in the level of expression of: any one of the kinA, phrA, and phrE genes; any two of the kinA, phrA, and phrE genes; or all three of the kinA, phrA, and phrE genes. In other embodiments, the genetic alteration results in a decrease in the activity of one or more of the KinA, PhrA, and PhrE proteins in the altered Gram positive bacterial cell as compared to a corresponding unaltered Gram positive bacterial cell grown under essentially the same culture conditions. Thus, the genetic alteration can results in a decrease in the activity of: any one of the KinA, PhrA, and PhrE proteins; any two of the KinA, PhrA, and PhrE proteins; or all three of the KinA, PhrA, and PhrE proteins.

In certain embodiments, the sequence of the wild type kinA gene is at least 60% identical to SEQ ID NO:1, the sequence of the wild type phrA gene is at least 60% identical to SEQ ID NO:6, and the sequence of the wild type phrE gene is at least 60% identical to SEQ ID NO:8. In certain embodiments, the sequence of the wild type KinA protein is at least 80% identical to SEQ ID NO:2, the sequence of the wild type PhrA protein is at least 80% identical to SEQ ID NO:7, and the sequence of the wild type PhrE protein is at least 80% identical to SEQ ID NO:9. In certain embodiments, the genetic alteration is a deletion of all or part of one or more of the kinA, phrA, and phrE genes.

In certain embodiments, the said altered Gram positive bacterial cell expresses a protein of interest. In certain embodiments, the method further comprises introducing an expression cassette encoding said protein of interest into said parental Gram positive bacterial cell. In certain embodiments, the method further comprises introducing an expression cassette encoding said protein of interest into said altered Gram positive bacterial cell. In certain embodiments, the protein of interest is a homologous protein. In certain embodiments, the protein of interest is a heterologous protein. In certain embodiments, the protein of interest is an enzyme. In certain embodiments, the enzyme is selected from the group consisting of: protease, cellulase, pullulanase, amylase, carbohydrase, lipase, isomerase, transferase, kinase, and phosphatase. In certain embodiments, the protein of interest is a protease. In certain embodiments, the protease is a subtilisin. In certain embodiments, the subtilisin is selected from the group consisting of: subtilisin 168, subtilisin BPN', subtilisin Carlsberg, subtilisin DY, subtilisin 147, subtilisin 309, and variants thereof.

In certain embodiments, the method further comprises culturing said altered Gram positive bacterial cell under conditions such that said protein of interest is expressed by said altered Gram positive bacterial cell. In certain embodiments, the method further comprises recovering said protein of interest.

Aspects of the present invention include altered Gram positive bacterial cell produced by the methods described above.

### BRIEF DESCRIPTION OF THE DRAWINGS

FIG. 1 shows a genetic map of the kinA (ΔkinA) deletion.

FIG. 2A shows a graph of cell densities of unaltered (parental) B. subtilis cells and altered (ΔkinA) B. subtilis cells expressing AmyE.

FIG. 2B shows a graph of AmyE expression from unaltered (parental) B. subtilis cells and altered (ΔkinA) B. subtilis cells.

FIG. 3A shows a graph of cell densities of unaltered (parental) B. subtilis cells and altered (ΔkinA) B. subtilis cells expressing FNA.

FIG. 3B shows a graph of FNA expression from unaltered (parental) B. subtilis cells and altered (ΔkinA) B. subtilis cells.

FIG. 4A shows a graph of cell densities of unaltered (parental) B. subtilis cells and altered (ΔkinA) B. subtilis cells expressing green fluorescent protein (GFP).

FIG. 4B shows a graph of GFP expression from unaltered (parental) B. subtilis cells and altered (ΔkinA) B. subtilis cells.

FIG. 5 shows a schematic representation of the phosphorelay pathway which regulates sporulation initiation in Bacillus cells. The auto phosphorylation of one or more kinases is triggered by a specific starvation signal, followed by the sequential phosphorylation of Spo0F, Spo0B and Spo0A proteins. Spo0A˜P controls the activation of the sporulation cascade. Kinases (e.g, kinA, kinB, KinC, kinD, kinE) and phosphatases (e.g., RapA, RapB, RapE) are indicated by gene names. Arrows indicate a positive effect such as phosphorylation or control over the expression of a target gene, while blunt-face lines indicate a negative effect such as dephosphorylation or repression of gene expression. For example, the kinase KinA phosphorylates the Spo0F phosphatase, that transfers the phosphoryl group to Spo0B and then Spo0A, while the transcriptional regulator AbrB inhibits spo0H (sigH) expression and consequently spo0A expression.

FIG. 6 shows a genetic construct of the phrA deletion.

FIG. 7 shows a genetic construct of the phrE deletion.

FIG. 8A shows a graph of cell densities of unaltered (parental) B. subtilis cells and altered B. subtilis cells (i.e., the altered B. subtilis cells comprise a deletion of both phrA and phrE genes; herein ΔphrA/ΔphrE) expressing GFP.

FIG. 8B shows a graph of GFP expression from unaltered (parental) B. subtilis cells and altered (ΔphrA/ΔphrE) B. subtilis cells.

FIG. 9A shows a graph of cell densities of unaltered (parental) B. subtilis cells and altered (ΔphrA/ΔphrE) B. subtilis cells expressing FNA.

FIG. 9B shows a graph of FNA expressionfrom unaltered (parental) B. subtilis cells and altered (ΔphrA/ΔphrE) B. subtilis cells.

FIG. 10A shows a graph of cell densities of unaltered (parental) B. subtilis cells and altered (ΔphrA/ΔphrE) B. subtilis cells expressing AmyE.

FIG. 10B shows a graph of AmyE expression from unaltered (parental) B. subtilis cells and altered (ΔphrA/ΔphrE) B. subtilis cells expressing.

### DETAILED DESCRIPTION OF THE INVENTION

The present invention relates in general to bacterial cells having a genetic alteration that results in increased expression/production of a protein of interest (hereinafter, a “POI”) and methods of making and using such cells. Certain aspects of the present invention include Gram positive microorganisms, such as members of the Bacillus genus, comprising a genetic alteration that reduces the expression and/or the activity of one or more proteins that activate the phosphorelay pathway, which results in increased expression of a POI.

Before the present compositions and methods are described in greater detail, it is to be understood that the present compositions and methods are not limited to particular embodiments described, as such may, of course, vary. It is also to be understood that the terminology used herein is for the purpose of describing particular embodiments only, and is not intended to be limiting, since the scope of the present compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference. The citation of any publication is for its disclosure prior to the filing date and should not be construed as an admission that the present compositions and methods are not entitled to antedate such publication by virtue of prior invention.

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element (e.g., such as a proviso). As such, this statement is intended to serve as antecedent basis for use of such exclusive terminology as “solely,” “only”, “excluding”,and the like in connection with the recitation of claim elements, or use of a “negative” limitation.

It is further noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) may further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

### Definitions

The present invention generally relates to Gram positive bacterial cells (and methods of making and using the same) that have been altered or modified to have an increased capacity to express and/or produce one or more POI.

Thus, certain embodiments are directed to altered Gram positive bacterial cells comprising at least one genetic alteration that reduces the expression of one or more genes that function to activate the phosphorelay pathway (e.g., genes encoding KinA, PhrA, PhrE). For example, the phosphorelay pathway (i.e., a signal transduction system) in B. subtilis is generally believed to revolve around the transcription factor Spo0A (see, “Bacillus subtilis and Other Gram-Positive Bacteria: Biochemistry, Physiology and Molecular Genetics”; Eds. A. L.Sonenshein, J. A. Hoch, R. Losick, Am. Society of Micorbiology, 1993).

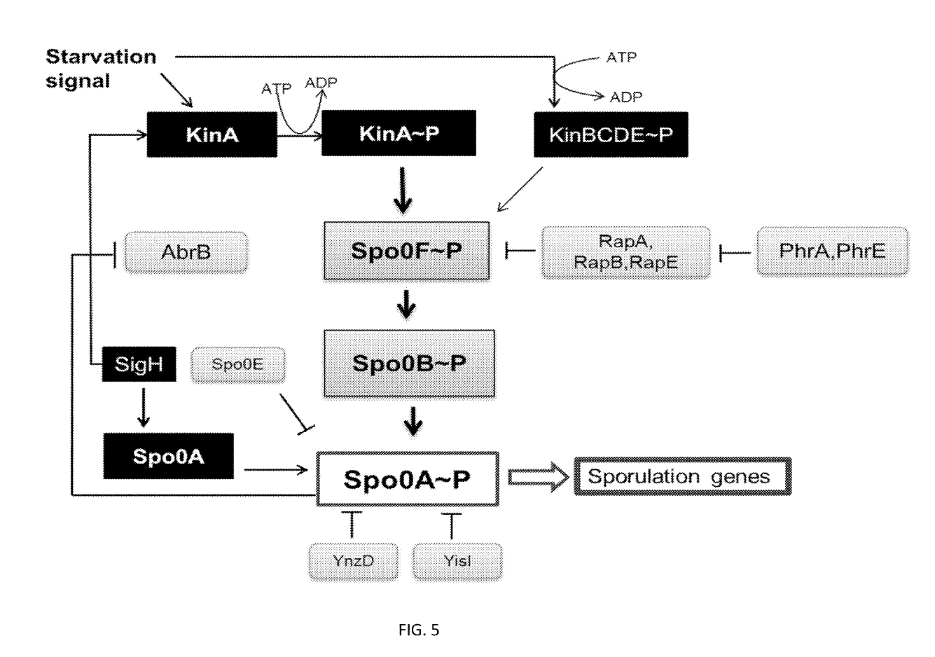

More particularly, it is believed that the role of the phosphorelay signal transduction system is to ultimately phosphorylate the (inactive) Spo0A transcription factor to Spo0A˜P, wherein the active Spo0A˜P transcription factor is responsible for transcription of genes involved in the initial-stages of sporulation. Without wishing to be bound by any particular theory or mode of operation of the instant invention, FIG. 5 generally shows a schematic representation of the phosphorelay pathway which regulates sporulation initiation in Bacillus cells. For example, certain kinases (e.g., KinA, KinB, etc.) are believed to be involved in interpreting environmental signals and transducing this information into auto-phosphorylated kinase proteins (e.g., KinA to KinA˜P). The phosphorylated kinases then transfer the phosphate to the Spo0F protein to generate Spo0F˜P, which is believed to serve as a secondary messenger in the phosphorelay, wherein Spo0F˜P transfers its phosphate to Spo0B to yield Spo0B˜P, which then transfer the phosphate group Spo0A to yield Spo0A˜P.

Without wishing to be bound or held to any particular theory, the Examples section set forth below demonstrates that blocking or reducing KinA activity (which blocks or reduces the phosphorylation and activation of the Spo0A transcription factor), results in increased expression of one or more a POIs in Bacillus cells. Furthermore, without wishing to be bound or held to any particular theory, the pentapeptide PhrA and the pentapeptide PhrE (see, FIG. 5) act to block the function of the RapA and RapE phosphatases, respectively, which de-represses the phosphorelay pathway activated by KinA. As demonstrated in the Example section, blocking the inhibitory activity of PhrA and/or PhrE on the Rap phosphatases results in increased expression of a POI in Bacillus cells.

As defined herein, an “altered cell”, a “modified cell”, an “altered bacterial cell”, a “modified bacterial cell”, an “altered host cell” or a “modified host cell” may be used interechangeably and refer to recombinant Gram positive bacterial cells that comprise at least one genetic alteration that reduces the expression of one or more genes that function to activate the phosphorelay pathway. For example, an “altered” Gram positive bacterial cell of the instant disclosure may be further defined as an “altered cell” which is derived from a parental bacterial cell, wherein the altered (daughter) cell comprises at least one genetic alteration that reduces expression of one or more genes that function to activate the phosphorelay pathway.

As defined herein, an “unaltered cell”, an “unmodified cell”, an “unaltered bacterial cell”, an “unmodified bacterial cell”, an “unaltered host cell” or an “unmodified host cell” may be used interechangeably and refer to “unaltered” ‘parental’ Gram positive bacterial cells that do not comprises the at least one genetic alteration that reduces the expression of one or more genes that function to activate the phosphorelay pathway. In certain embodiments, an unaltered (parental) Gram positive bacterial cell is refered to as a “control cell” or an unaltered (parental) Gram positive bacterial “control” cell.

For example, certain embodiments of the disclosure are directed to “altered” Gram positive bacterial (daughter) cells expressing an increased amount of a POI, wherein the increased amount of the POI is relative to the expression of the same POI in an “unaltered” Gram positive bacterial (parental) cells (i.e., an unaltered Gram positive bacterial “control” cell. Thus, as defined herein, when the terms or phrases “unaltered bacterial cell(s)”, “unaltered Gram positive bacterial cell(s)”, “unaltered Gram positive bacterial ‘control’ cell(s)” and the like are used in the context of comparison to the one or more “altered bacterial cells” of the disclosure, it is understood that both the altered (daughter) cells and the unaltered parental (control) cells are grown/cultured under essentially identitical conditions and media.

As used herein, “host” cell refers to a “Gram positive bacterial cell” that has the capacity to act as a host or expression vehicle for a newly introduced DNA sequence.

In certiain embodiments of the present invention,a host cell is a member of the

Bacillus genus.

As used herein, “the genus Bacillus” or “Bacillus sp.” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii, and B. thuringiensis.

It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

As used herein, the term “vector” refers to any nucleic acid that can be replicated in cells and can carry new genes or DNA (polynucleotide) segments into cells. Thus, the term refers to a nucleic acid construct designed for transfer between different host cells. An “expression vector” refers to a vector that has the ability to express heterologous DNA fragments in a foreign cell. Many prokaryotic and eukaryotic expression vectors are commercially available. A “targeting vector” is a vector that includes polynucleotide sequences that are homologus to a region in the choromosome of a host cell into which it is transformed and that can drive homologous recombination at that region. Targetting vectors find use in introducing mutations into the chromosome of a cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. Selection and/or construction of appropriate vector(s) is within the knowledge of those having skill in the art.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

By “purified” or “isolated” or “enriched” is meant that a biomolecule (e.g., a polypeptide or polynucleotide) is altered from its natural state by virtue of separating it from some or all of the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, the terms “increased”, “enhanced” and “improved”, when referring to expression of a biomolecule of interest (e.g., a protein on interest), are used interchangeably herein to indicate that expression of the biomolecule (i.e., in the altered cell) is above the level of expression in a corresponding unaltered (parental) cell that has been grown under essentially the same growth conditions.

As defined herein, term “expression” or “expressed” with respect to a gene sequence, an ORF sequence or polynucleotide sequence, refers to transcription of the gene, ORF or polynucleotide and, as appropriate, translation of the resulting mRNA transcript to a protein. Thus, as will be clear from the context, expression of a protein results from transcription and translation of the open reading frame sequence. The level of expression of a desired product in a host microorganism may be determined on the basis of either the amount of corresponding mRNA that is present in the host, or the amount of the desired product encoded by the selected sequence. For example, mRNA transcribed from a selected sequence can be quantitated by PCR or by northern hybridization (see Sambrook et al., Molecular Cloning: A Laboratory Manual, Cold Spring Harbor Laboratory Press, 1989). Protein encoded by a selected sequence can be quantitated by various methods (e.g., by ELISA, by assaying for the biological activity of the protein, or by employing assays that are independent of such activity, such as western blotting or radioimmunoassay, using antibodies that are recognize and bind reacting the protein). the term “expression” in the context of a gene (or polynucleotide thereof), is the process by which a protein is produced based on the nucleic acid sequence of the gene (or polynucleotide thereof), and thus includes both transcription and translation.

As defined herein, the term “introducing”, as used in phrases such as “introducing into the bacterial cell” at least one polynucleotide open reading frame (ORF), or a gene thereof, or a vector thereof, includes methods known in the art for introducing polynucleotides into a cell, including, but not limited to protoplast fusion, transformation (e.g., calcium chloride, electroporation), transduction, transfection, conjugation and the like (see e.g., Ferrari et al., “Genetics,” in Hardwood et al, (eds.), Bacillus, Plenum Publishing Corp., pages 57-72, 1989).

As used herein, the terms “transformed” and “stably transformed” refers to a cell into which a polynucleotide sequence has been introduced by human intervention. The polynucleotide can be integrated into the genome of the cell or be present as an episomal plasmid that is maintained for at least two generations.

As used herein, the terms “selectable marker” or “selective marker” refer to a nucleic acid (e.g., a gene) capable of expression in host cell which allows for ease of selection of those hosts containing the nucleic acid. Examples of such selectable markers include but are not limited to antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation. Other markers useful in accordance with the invention include, but are not limited to auxotrophic markers, such as tryptophan; and detection markers, such as β-galactosidase.

As used herein, the term “promoter” refers to a nucleic acid sequence that functions to direct transcription of a downstream gene. In embodiments, the promoter is appropriate to the host cell in which the target gene is being expressed. The promoter, together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

As used herein, “functionally attached” or “operably linked” means that a regulatory region or functional domain having a known or desired activity, such as a promoter, terminator, signal sequence or enhancer region, is attached to or linked to a target (e.g., a gene or polypeptide) in such a manner as to allow the regulatory region or functional domain to control the expression, secretion or function of that target according to its known or desired activity.

The term “genetic alteration” when used to describe a recombinant cell (e.g., an “altered” Gram positive bacterial cell) means that the cell has at least one genetic difference as compared to the parental cell. The one or more genetic alterations may be a chromosomal mutation (e.g., an insertion, a deletion, substitution, inversion, replacement of a chromosomal region with another (e.g., replacement of a chromosomal promoter with a heterologous promoter), etc.) and/or the introduction of an extra-chromosomal polynucleotide (e.g., a plasmid). In some embodiments, an extra-chormosomal polynucleotide may be integrated into the chromosome of the host cell to generate a stable transfectant/transformant. Embodiments of the present disclosure include genetic alterations that reduce the expression or activity of the KinA, PhrA, and/or PhrE proteins (either transcriptionally, translationally, or by reducing the activity of the protein itself e.g., by mutation of the amino acid sequence). As detailed herein, such alterations improve the expression of proteins of interest.

“Inactivation” of a gene means that the expression of a gene, or the activity of its encoded protein, is blocked or is otherwise unable to exert its known function. Inactivation of a gene can be performed via any suitable means, e.g., via a genetic alteration as described above. In certain embodiments, the expression product of an inactivated gene is a truncated protein with a corresponding change in the biological activity of the protein. In some embodiments, an altered Gram positive bacterial cell comprises inactivation of one or more genes that results in stable and non-reverting inactivation.

In some embodiments, gene inactivation is achieved by deletion. In some embodiments, the region targeted for deletion (e.g., a gene) is deleted by homologous recombination. For example, a DNA construct comprising an incoming sequence having a selective marker flanked on each side by sequences that are homologous to the region targeted for deletion is used (where the sequences may be referred to herein as a “homology box”). The DNA construct aligns with the homologous sequences of the host chromosome and in a double crossover event the region targeted for deletion is excised out of the host cell chromosome.

An “insertion” or “addition” is a change in a nucleotide or amino acid sequence which has resulted in the addition of one or more nucleotides or amino acid residues, respectively, as compared to the naturally occurring or parental sequence.

As used herein, a “substitution” results from the replacement of one or more nucleotides or amino acids by different nucleotides or amino acids, respectively.

Methods of mutating genes are well known in the art and include but are not limited to site-directed mutation, generation of random mutations, and gapped-duplex approaches (See e.g., U.S. Pat. No. 4,760,025; Moring et al., Biotech. 2:646 [1984]; and Kramer et al., Nucleic Acids Res., 12:9441 [1984]).

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, “ortholog” and “orthologous genes” refer to genes in different species that have evolved from a common ancestral gene (i.e., a homologous gene) by speciation. Typically, orthologs retain the same function in during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, “homology” refers to sequence similarity or identity, with identity being preferred. This homology is determined using standard techniques known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

As used herein, an “analogous sequence” is one wherein the function of the gene is essentially the same as the gene designated from Bacillus subtilis strain 168. Additionally, analogous genes include at least 60%, 65%, 70%, 75%, 80%, 85%, 90%, 95%, 97%, 98%, 99% or 100% sequence identity with the sequence of the Bacillus subtilis strain 168 gene. Alternately, analogous sequences have an alignment of between 70 to 100% of the genes found in the B. subtilis 168 region and/or have at least between 5 - 10 genes found in the region aligned with the genes in the B. subtilis 168 chromosome. In additional embodiments more than one of the above properties applies to the sequence. Analogous sequences are determined by known methods of sequence alignment. A commonly used alignment method is BLAST, although as indicated above and below, there are other methods that also find use in aligning sequences.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle (Feng and Doolittle, J. Mol. Evol., 35:351-360 [1987]). The method is similar to that described by Higgins and Sharp (Higgins and Sharp, CABIOS 5:151-153 [1989]). Useful PILEUP parameters including a default gap weight of 3.00, a default gap length weight of 0.10, and weighted end gaps.

Another example of a useful algorithm is the BLAST algorithm, described by Altschul et al., (Altschul et al., J. Mol. Biol., 215:403-410, [1990]; and Karlin et al., Proc. Natl. Acad. Sci. USA 90:5873-5787 [1993]). A particularly useful BLAST program is the WU-BLAST-2 program (See, Altschul et al., Meth. Enzymol., 266:460-480 [1996]).

As used herein, “percent (%) sequence identity” with respect to the amino acid or nucleotide sequences identified herein is defined as the percentage of amino acid residues or nucleotides in a candidate sequence that are identical with the amino acid residues or nucleotides in a sequence of interest, after aligning the sequences and introducing gaps, if necessary, to achieve the maximum percent sequence identity, and not considering any conservative substitutions as part of the sequence identity.

By “homologue” (or “homolog”) shall mean an entity having a specified degree of identity with the subject amino acid sequences and the subject nucleotide sequences. A homologous sequence is can include an amino acid sequence that is at least 60%, 65%, 70%, 75%, 80%, 85%, 86%, 87%, 88%, 89%, 90%, 91%, 92%, 93%, 94%, 95%, 96%, 97%, 98% or even 99% identical to the subject sequence, using conventional sequence alignment tools (e.g., Clustal, BLAST, and the like). Typically, homologues will include the same active site residues as the subject amino acid sequence, unless otherwise specified.

Methods for performing sequence alignment and determining sequence identity are known to the skilled artisan, may be performed without undue experimentation, and calculations of identity values may be obtained with definiteness. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Protein Sequence and Structure 5:Suppl. 3 (National Biomedical Research Foundation, Washington, D.C.). A number of algorithms are available for aligning sequences and determining sequence identity and include, for example, the homology alignment algorithm of Needleman et al. (1970) J. Mol. Biol. 48:443; the local homology algorithm of Smith et al. (1981) Adv. Appl. Math. 2:482; the search for similarity method of Pearson et al. (1988) Proc. Natl. Acad. Sci. 85:2444; the Smith-Waterman algorithm (Meth. Mol. Biol. 70:173-187 (1997); and BLASTP, BLASTN, and BLASTX algorithms (see Altschul et al. (1990) J. Mol. Biol. 215:403-410).

Computerized programs using these algorithms are also available, and include, but are not limited to: ALIGN or Megalign (DNASTAR) software, or WU-BLAST-2 (Altschul et al., Meth. Enzym., 266:460-480 (1996)); or GAP, BESTFIT, BLAST, FASTA, and TFASTA, available in the Genetics Computing Group (GCG) package, Version 8, Madison, Wis., USA; and CLUSTAL in the PC/Gene program by Intelligenetics, Mountain View, Calif. Those skilled in the art can determine appropriate parameters for measuring alignment, including algorithms needed to achieve maximal alignment over the length of the sequences being compared.

As used herein, the term “hybridization” refers to the process by which a strand of nucleic acid joins with a complementary strand through base pairing, as known in the art.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while anintermediate or low stringency hybridization can be used to identify or detect polynucleotide sequence homologs.

Moderate and high stringency hybridization conditions are well known in the art. An example of high stringency conditions includes hybridization at about 42° C. in 50% formamide, 5× SSC, 5× Denhardt's solution, 0.5% SDS and 100 μg/ml denatured carrier DNA followed by washing two times in 2× SSC and 0.5% SDS at room temperature and two additional times in 0.1× SSC and 0.5% SDS at 42° C. An example of moderate stringent conditions include an overnight incubation at 37° C. in a solution comprising 20% formamide, 5× SSC (150 mM NaCl, 15 mM trisodium citrate), 50 mM sodium phosphate (pH 7.6), 5 × Denhardt's solution, 10% dextran sulfate and 20 mg/ml denaturated sheared salmon sperm DNA, followed by washing the filters in 1× SSC at about 37 - 50° C. Those of skill in the art know how to adjust the temperature, ionic strength, etc. as necessary to accommodate factors such as probe length and the like.

The term “recombinant,” when used in reference to a biological component or composition (e.g., a cell, nucleic acid, polypeptide/enzyme, vector, etc.) indicates that the biological component or composition is in a state that is not found in nature. In other words, the biological component or composition has been modified by human intervention from its natural state. For example, a recombinant cell encompass a cell that expresses one or more genes that are not found in its native parent (i.e., non-recombinant) cell, a cell that expresses one or more native genes in an amount that is different than its native parent cell, and/or a cell that expresses one or more native genes under different conditions than its native parent cell. Recombinant nucleic acids may differ from a native sequence by one or more nucleotides, be operably linked to heterologous sequences (e.g., a heterologous promoter, a sequence encoding a non-native or variant signal sequence, etc.), be devoid of intronic sequences, and/or be in an isolated form. Recombinant polypeptides/enzymes may differ from a native sequence by one or more amino acids, may be fused with heterologous sequences, may be truncated or have internal deletions of amino acids, may be expressed in a manner not found in a native cell (e.g., from a recombinant cell that over-expresses the polypeptide due to the presence in the cell of an expression vector encoding the polypeptide), and/or be in an isolated form. It is emphasized that in some embodiments, a recombinant polynucleotide or polypeptide/enzyme has a sequence that is identical to its wild-type counterpart but is in a non-native form (e.g., in an isolated or enriched form).

As used herein, the term “target sequence” refers to a DNA sequence in the host cell that encodes the sequence where it is desired for the incoming sequence to be inserted into the host cell genome. In some embodiments, the target sequence encodes a functional wild-type gene or operon, while in other embodiments the target sequence encodes a functional mutant gene or operon, or a non-functional gene or operon.

As used herein, a “flanking sequence” refers to any sequence that is either upstream or downstream of the sequence being discussed (e.g., for genes A-B-C, gene B is flanked by the A and C gene sequences). In a embodiment, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In a embodiment, the 5′ and 3′ ends of a selective marker are flanked by a polynucleotide sequence comprising a section of the inactivating chromosomal segment. In some embodiments, a flanking sequence is present on only a single side (either 3′ or 5′), while in embodiments, it is present on each side of the sequence being flanked.

As used herein, the terms “amplifiable marker,” “amplifiable gene,” and “amplification vector” refer to a gene or a vector encoding a gene which permits the amplification of that gene under appropriate growth conditions.

“Template specificity” is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (See e.g., Kacian et al., Proc. Natl. Acad. Sci. USA 69:3038 [1972]). Other nucleic acids are not replicated by this amplification enzyme. Similarly, in the case of T7 RNA polymerase, this amplification enzyme has a stringent specificity for its own promoters (See, Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (See, Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences.

As used herein, the term “amplifiable nucleic acid” refers to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of “target” (defined below). In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the methods of U.S. Pat. Nos. 4,683,195 4,683,202, and 4,965,188, hereby incorporated by reference, which include methods for increasing the concentration of a segment of a target sequence in a mixture of genomic DNA without cloning or purification.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “RT-PCR” refers to the replication and amplification of RNA sequences. In this method, reverse transcription is coupled to PCR, most often using a one enzyme procedure in which a thermostable polymerase is employed, as described in U.S. Pat. No. 5,322,770, herein incorporated by reference. In RT-PCR, the RNA template is converted to cDNA due to the reverse transcriptase activity of the polymerase, and then amplified using the polymerizing activity of the polymerase (i.e., as in other PCR methods).

As used herein, the term “chromosomal integration” refers to the process whereby the incoming sequence is introduced into the chromosome of a host cell (e.g., Bacillus). The homologous regions of the transforming DNA align with homologous regions of the chromosome. Subsequently, the sequence between the homology boxes is replaced by the incoming sequence in a double crossover (i.e., homologous recombination). In some embodiments of the present invention, homologous sections of an inactivating chromosomal segment of a DNA construct align with the flanking homologous regions of the indigenous chromosomal region of the Bacillus chromosome. Subsequently, the indigenous chromosomal region is deleted by the DNA construct in a double crossover (i.e., homologous recombination).

“Homologous recombination” means the exchange of DNA fragments between two DNA molecules or paired chromosomes at the site of identical or nearly identical nucleotide sequences. In a embodiment, chromosomal integration is homologous recombination.

“Homologous sequences” as used herein means a nucleic acid or polypeptide sequence having 100%, 99%, 98%, 97%, 96%, 95%, 94%, 93%, 92%, 91%, 90%, 88%, 85%, 80%, 75%, or 70% sequence identity to another nucleic acid or polypeptide sequence when optimally aligned for comparison. In some embodiments, homologous sequences have between 85% and 100% sequence identity, while in other embodiments there is between 90% and 100% sequence identity, and in more embodiments, there is 95% and 100% sequence identity.

As used herein “amino acid” refers to peptide or protein sequences or portions thereof. The terms “protein”, “peptide” and “polypeptide” are used interchangeably.

As used herein, “protein of interest” (POI) refers to a protein/polypeptide that is desired and/or being assessed. In some embodiments, the protein of interest is intracellular, while in other embodiments, it is a secreted polypeptide. Polypeptides include enzymes, including, but not limited to those selected from amylolytic enzymes, proteolytic enzymes, cellulytic enzymes, oxidoreductase enzymes and plant cell-wall degrading enzymes. More particularly, these enzyme include, but are not limited to amylases, proteases, xylanases, lipases, laccases, phenol oxidases, oxidases, cutinases, cellulases, hemicellulases, esterases, peroxidases, catalases, glucose oxidases, phytases, pectinases, perhydrolases, polyol oxidases, pectate lyases, glucosidases, isomerases, transferases, galactosidases and chitinases. In particular embodiments of the present invention, the polypeptide of interest is a protease. In some embodiments, the protein of interest is a secreted polypeptide which is fused to a signal peptide (i.e., an amino-terminal extension on a protein to be secreted). Nearly all secreted proteins use an amino-terminal protein extension which plays a crucial role in the targeting to and translocation of precursor proteins across the membrane. This extension is proteolytically removed by a signal peptidase during or immediately following membrane transfer.

In some embodiments of the present invention, the polypeptide of interest is selected from hormones, antibodies, growth factors, receptors, etc. Hormones encompassed by the present invention include but are not limited to, follicle-stimulating hormone, luteinizing hormone, corticotropin-releasing factor, somatostatin, gonadotropin hormone, vasopressin, oxytocin, erythropoietin, insulin and the like. Growth factors include, but are not limited to platelet-derived growth factor, insulin-like growth factors, epidermal growth factor, nerve growth factor, fibroblast growth factor, transforming growth factors, cytokines, such as interleukins (e.g., IL-1 through IL-13), interferons, colony stimulating factors, and the like. Antibodies include but are not limited to immunoglobulins obtained directly from any species from which it is desirable to produce antibodies. In addition, the present invention encompasses modified antibodies. Polyclonal and monoclonal antibodies are also encompassed by the present invention. In particularly embodiments, the antibodies are human antibodies.

As used herein, a “derivative” or “variant” of a polypeptide means a polypeptide, which is derived from a precursor polypeptide (e.g., the native polypeptide) by addition of one or more amino acids to either or both the C- and N-terminal ends, substitution of one or more amino acids at one or a number of different sites in the amino acid sequence, deletion of one or more amino acids at either or both ends of the polypeptide or at one or more sites in the amino acid sequence, insertion of one or more amino acids at one or more sites in the amino acid sequence, and any combination thereof. The preparation of a derivative or variant of a polypeptide may be achieved in any convenient manner, e.g., by modifying a DNA sequence which encodes the native polypeptides, transformation of that DNA sequence into a suitable host, and expression of the modified DNA sequence to form the derivative/variant polypeptide. Derivatives or variants further include polypeptides that are chemically modified.

As used herein, the term “heterologous protein” refers to a protein or polypeptide that does not naturally occur in the host cell. Examples of heterologous proteins include enzymes such as hydrolases including proteases, cellulases, amylases, carbohydrases, and lipases; isomerases such as racemases, epimerases, tautomerases, or mutases; transferases, kinases and phophatases. In some embodiments, the proteins are therapeutically significant proteins or peptides, including but not limited to growth factors, cytokines, ligands, receptors and inhibitors, as well as vaccines and antibodies. In additional embodiments, the proteins are commercially important industrial proteins/peptides (e.g., proteases, carbohydrases such as amylases and glucoamylases, cellulases, oxidases and lipases). In some embodiments, the genes encoding the proteins are naturally occurring genes, while in other embodiments, mutated and/or synthetic genes are used.

As used herein, “homologous protein” refers to a protein or polypeptide native or naturally occurring in a cell. In certain embodiments, the cell is a Gram positive cell, while in certain other embodiments the Gram positive cell is a Bacillus cell. In alternative embodiments, the homologous protein is a native protein produced by other organisms, including but not limited to E. coli. The invention encompasses host cells producing the homologous protein via recombinant DNA technology.

As used herein, an “operon” comprises a group of contiguous genes that can be transcribed as a single transcription unit from a common promoter, and are thereby subject to co-regulation. In some embodiments, an operon may include multiple promoters that drive the transcription of multiple different mRNAs.

As set forth above, certain embodiments of the present disclosure relate to altered bacterial cells comprising a genetic alteration that results in the increased expression of a POI and methods of making and using such cells. Thus, certain aspects of the present invention include altered Gram positive cells, such as members of the Bacillus genus, wherein the altered Gram positive bacterial (daughter) cells comprise a genetic alteration that results in a decrease in the level of expression of at least one gene selected from a kinA gene, a phrA gene and/or a phrE gene. As set forth herein, and further described in the Examples section, the altered Gram positive bacterial cells of the instant invention (i.e., comprising a genetic alteration that results in a decrease in the level of expression of at least one gene selected from a kinA gene, a phrA gene and/or a phrE gene) demonstrate increased expression of one or more POIs, when compared to a corresponding unaltered Gram positive bacterial (parental) cell grown under essentially the same culture conditions. Thus, a genetic alteration of the present disclosure is any alteration which decreases the level of expression of any one of the kinA, phrA and phrE genes; any two of the kinA, phrA and phrE genes; or all three of the kinA, phrA, and phrE genes. In other embodiments, the genetic alteration results in a decrease in the activity of one or more of the KinA, PhrA, and PhrE proteins in the altered Gram positive bacterial (daughter) cell as compared to a corresponding unaltered Gram positive bacterial (parental) cell grown under essentially the same culture conditions. Thus, in certain embodiments, a genetic alteration is any alteration which decreases the activity of any one of the KinA, PhrA, and PhrE proteins; any two of the KinA, PhrA, and PhrE proteins; or all three of the KinA, PhrA, and PhrE proteins.

As summarized above, aspects of the invention include methods for increasing expression of a POI from a Gram positive bacterial cell and is based on the observation that the production of a POI is increased in Gram positive (daughter) cells that have been genetically altered to have reduced expression of one or more genes that activate the phosphorelay pathway,which is relative to the production of the same POI in a corresponding unaltered Gram positive (parental) cell. As set forth above, a genetic alteration is defined as any alteration in a host cell that changes the genetic make-up of the host cell, for example by episomal addition and/or chromosomal insertion, deletion, inversion, base change, etc. No limitation in this regard is intended.

In certain embodiments, the parental Gram positive cell has one or more defective or inactive sporulation-initiating genes (i.e., genes whose expression is controlled by Spo0A or downstream of Spo0A), and thus the parental cell is prevented from forming spores. Surprisingly, Applicants of the instant invention found that even in this genetic background (i.e., a parental Gram positive cell comprising one or more defective or inactive sporulation-initiating genes), the additional genetic alterations (e.g., a genetic alteration that results in a decrease in the level of expression of at least one gene selected from a kinA gene, a phrA gene and/or a phrE gene)) increased the expression of POIs in such altered Gram positive bacterial (daughter) cells. Therefore, the improvement in protein expression/production in the genetically altered (daughter) cells of the disclosure is not due solely to preventing sporulation of the Gram positive cell. For example, the parental Gram positive cell from which the altered Gram positive cell of the disclosure is derived can have a non-functional/mutated/deleted sporulation gene regulated by Spo0A or by the Sigma factors SigF, SigG, SigE and SigK (e.g., see Examples section, which employ sporulation deficient Bacillus cells).

In certain embodiments, the invention is directed to methods (and compositions thereof) for producing or obtaining an altered Gram positive bacterial (daughter) cell comprising at least one genetic alteration that reduces expression of one or more genes that activate the phosphorelay pathway. In other embodiments, the altered altered Gram positive bacterial (daughter) cell comprising at least one genetic alteration that reduces expression of one or more genes that activate the phosphorelay pathway expresses and/or produces an increased amount of one or more POIwhen cultured under conditions such that the protein of interest is expressed by the altered Gram positive (daughter) bacterial cell. Thus, the expression and/or production of the POI is thereby increased in the altered Gram positive bacterial (daughter) cell when compared (i.e., relative) to the expression and/or production of the same POI in a corresponding unaltered Gram positive bacterial (parental) cell grown under essentially the same culture conditions.

According to certain embodiments, the genetically altered Gram positive bacterial cell (or parental cell from which the genetically altered Gram positive bacterial cell is produced) is a member of the Bacillus genus. In some embodiments, the Bacillus cell is alkalophilic Bacillus cell. Numerous alkalophilic Bacillus cells are known in the art (See e.g., U.S. Pat. No. 5,217,878; and Aunstrup et al., Proc IV IFS: Ferment. Technol. Today, 299-305 [1972]). In some embodiments, the Bacillus cell is an industrial relevant Bacillus cell. Examples of industrial Bacillus cells include, but are not limited to B. licheniformis, B. lentus, B. subtilis, and B. amyloliquefaciens. In additional embodiments, the Bacillus cell is selected from the group consisting of B. licheniformis, B. lentus, B. subtilis, B. amyloliquefaciens, B. brevis, B. stearothermophilus, B. alkalophilus, B. coagulans, B. circulans, B. pumilus, B. lautus, B. clausii, B. megaterium, or B. thuringiensis, as well as other organisms within the genus Bacillus, as discussed above. In particular embodiments, a B. subtilis cell is used. For example, U.S. Pat. Nos. 5,264,366 and 4,760,025 (RE 34,606) describe various Bacillus host cells that find use in the present invention, although other suitable cells are contemplated for use in the present invention.

The parental cell of a genetically altered Gram positive cell as described herein (e.g., a parental Bacillus cell) is a recombinant Gram positive cell wherein a heterologous polynucleotide encoding a POI has been introduced into the cell. While the introduction of a polynucleotide encoding a POI may be done in a parental cell, this step may also be performed in a cell that has already been genetically altered for increased polypeptide production as detailed herein. In some embodiments, the host cell is a Bacillus subtilis host strain, e.g., a recombinant B. subtilis host strain.

Numerous B. subtilis strains are known that find use in aspects of the present invention, including but not limited to 1A6 (ATCC 39085), 168 (1A01), SB19, W23, Ts85, B637, PB1753 through PB1758, PB3360, JH642, 1A243 (ATCC 39,087), ATCC 21332, ATCC 6051, M1113, DE100 (ATCC 39,094), GX4931, PBT 110, and PEP 211strain (See e.g., Hoch et al., Genetics, 73:215-228 [1973]; U.S. Pat. No. 4,450,235; U.S. Pat. No. 4,302,544; and EP 0134048). The use of B. subtilis as an expression host is further described by Palva et al. and others (See, Palva et al., Gene 19:81-87 [1982]; also see Fahnestock and Fischer, J. Bacteriol., 165:796-804 [1986]; and Wang et al, Gene 69:39-47 [1988]).

In certain embodiments, industrial protease producing Bacillus strains can serve as parental expression hosts. In some embodiments, use of these strains in the present invention provides further enhancements in efficiency and protease production. Two general types of proteases are typically secreted by Bacillus sp., namely neutral (or “metalloproteases”) and alkaline (or “serine”) proteases. Serine proteases are enzymes which catalyze the hydrolysis of peptide bonds in which there is an essential serine residue at the active site. Serine proteases have molecular weights in the 25,000 to 30,000 range (See, Priest, Bacteriol. Rev., 41:711-753 [1977]). Subtilisin is a serine protease for use in the present invention. A wide variety of Bacillus subtilisins have been identified and sequenced, for example, subtilisin 168, subtilisin BPN', subtilisin Carlsberg, subtilisin DY, subtilisin 147 and subtilisin 309 (See e.g., EP 414279 B; WO 89/06279; and Stahl et al., J. Bacteriol., 159:811-818 [1984]). In some embodiments of the present invention, the Bacillus host strains produce mutant (e.g., variant) proteases. Numerous references provide examples of variant proteases (See e.g., WO 99/20770; WO 99/20726; WO 99/20769; WO 89/06279; RE 34,606; U.S. Pat. No. 4,914,031; U.S. Pat. No. 4,980,288; U.S. Pat. No. 5,208,158; U.S. Pat. No. 5,310,675; U.S. Pat. No. 5,336,611; U.S. Pat. No. 5,399,283; U.S. Pat. No. 5,441,882; U.S. Pat. No. 5,482,849; U.S. Pat. No. 5,631,217; U.S. Pat. No. 5,665,587; U.S. Pat. No. 5,700,676; U.S. Pat. No. 5,741,694; U.S. Pat. No. 5,858,757; U.S. Pat. No. 5,880,080; U.S. Pat. No. 6,197,567; and U.S. Pat. No. 6,218,165).

It is noted here that the present invention is not limited to proteases as the protein of interest. Indeed, the present disclosure encompasses a wide variety of proteins of interest for which increased expression in the Gram positive cell is desired (detailed below).

In other embodiments, a Gram positive bacterial cell for use in aspects of the present invention may have additional genetic alterations in other genes that provide beneficial phenotypes. For example, a Bacilluscell that includes a mutation or deletion in at least one of the following genes, degU, degS, degR and degQ may be employed. In some embodiments, the mutation is in a degU gene, e.g., a degU(Hy)32 mutation. (See, Msadek et al., J. Bacteriol., 172:824-834 [1990]; and Olmos et al., Mol. Gen. Genet., 253:562-567 [1997]). Thus, one example of a parental/genetically altered Gram positive cell that finds use in aspects of the present invention is a Bacillus subtilis cell carrying a degU32(Hy) mutation. In a further embodiment, the Bacillus host may include a mutation or deletion in scoC4, (See, Caldwell et al., J. Bacteriol., 183:7329-7340 [2001]); spoIIE (See, Arigoni et al., Mol. Microbiol., 31:1407-1415 [1999]); oppA or other genes of the opp operon (See, Perego et al., Mol. Microbiol., 5:173-185 [1991]). Indeed, it is contemplated that any mutation in the opp operon that causes the same phenotype as a mutation in the oppA gene will find use in some embodiments of the altered Bacillus cellsof the present invention. In some embodiments, these mutations occur alone, while in other embodiments, combinations of mutations are present. In some embodiments, an altered Bacillus of the invention is obtained from a parental Bacillus host strain that already includes a mutation to one or more of the above-mentioned genes. In alternate embodiments, a previously genetically altered Bacillus of the invention is further engineered to include mutation of one or more of the above-mentioned genes.

As indicated above, expression of at least one gene that activates the phosphorelay pathway is reduced in the genetically altered Gram positive cell as compared to a parental cell (grown under essentially the same conditions). This reduction of expression can be achieced in any convenient manner, and may be at the level of transcription, mRNA stability, translation, or may be due to the presence of a varation in one or more of the polypeptides produced from such genes that reduces its activity (i.e., it is a “functional” reduction of expression based on activity of the polypeptide). As such, no limitation in the type of genetic alteration or the manner through which expression of at least one gene that induces the expression of sporulation-initiating genes is reduced is intended. For example, in some embodiments the genetic alteration in the Gram positive cell is one that alters one or more of the promoters of the genes of interest, resulting in reduced transcriptional activity.

In certain embodiments, the genetic alteration results in a decrease in the level of expression of one or more of the kinA, phrA, and phrE genes in the altered Gram positive bacterial cell as compared to a corresponding unaltered Gram positive bacterial cell. Thus, the genetic alteration can result in a decrease in the level of expression of any one of the kinA, phrA, and phrE genes; any two of the kinA, phrA, and phrE genes; or all three of the kinA, phrA, and phrE genes. In other embodiments, the genetic alteration results in a decrease in the activity of one or more of the KinA, PhrA, and PhrE proteins in the altered Gram positive bacterial cell as compared to a corresponding unaltered Gram positive bacterial cell. Thus, the genetic alteration can results in a decrease in the activity of: any one of the KinA, PhrA, and PhrE proteins; any two of the KinA, PhrA, and PhrE proteins; or all three of the KinA, PhrA, and PhrE proteins.

In certain embodiments, the expression of the genes in the phosphorelay pathway for activating the expression of sporulation-initiating genes is reduced in the genetically altered Gram positive cell to about 3% of the level of expression in the wildtype and/or parental cell cultured under essentailly the same culture conditions, including about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 65%, about 70%, about 75%, or about 80%. As such, the range of reduction of expression of the one or more genes that induce the expression of sporulation-initiating genes can be from about 3% to about 80%, from about 4% to about 75%, from about 5% to about 70%, from about 6% to about 65%, from about 7% to about 60%, from about 8% to about 50%, from about 9% to about 45%, from about 10% to about 40%, from about 11% to about 35%, from about 12% to about 30%, from about 13% to about 25%, from about 14% to about 20%, etc. Any sub-range of expression within the ranges set forth above is contemplated.

In certain embodiments, the altered Gram positive bacterial cell has reduced expression of any one, two or three of the kinA, phrA, and phrE genes as compared to the expression of these genes in a corresponding unaltered Gram positive bacterial cell grown under essentially the same culture conditions.

In certain embodiments, the genetic alteration (or mutation) is one that reduces the expression of the kinA gene. A kinA gene in a parental Gram positive cell (i.e., prior to being genetically altered as described herein) is a gene that is at least 60% identical to SEQ ID NO:1, including at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO:1. In certain embociments, the genetic alteration is a deletion of all or a part of the kinA gene.

In certain embodiments, the genetic alteration (or mutation) is one that reduces the expression of the phrA gene. A phrA gene in a parental Gram positive cell (i.e., prior to being genetically altered as described herein) is a gene that is at least 60% identical to SEQ ID NO:6, including at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO:6. In certain embociments, the genetic alteration is a deletion of all or a part of the phrA gene.

In certain embodiments, the genetic alteration (or mutation) is one that reduces the expression of the phrE gene. A phrE gene in a parental Gram positive cell (i.e., prior to being genetically altered as described herein) is a gene that is at least 60% identical to SEQ ID NO:8, including at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO:8. In certain embociments, the genetic alteration is a deletion of all or a part of the phrE gene.

In certain embodiments, the altered Gram positive bacterial cell has reduced expression of any one, two or three of the kinA, phrA, and phrE genes as compared to the expression of these genes in a corresponding unaltered Gram positive bacterial cell grown under essentially the same culture conditions.

In certain embodiments, the genetic alteration (or mutation) is one that reduces the activity of the KinA protein, e.g., a variant KinA protein (e.g., having a deletion, insertion or substitution of one or more amino acids as compared to the wild type sequence). A variant KinA protein can contain an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99 identical to SEQ ID NO:2.

In certain embodiments, the genetic alteration (or mutation) is one that reduces the activity of the PhrA protein, e.g., a variant PhrA protein (e.g., having a deletion, insertion or substitution of one or more amino acids as compared to the wild type sequence). A variant PhrA protein can contain an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99 identical to SEQ ID NO:7.

In certain embodiments, the genetic alteration (or mutation) is one that reduces the activity of the PhrE protein, e.g., a variant PhrE protein (e.g., having a deletion, insertion or substitution of one or more amino acids as compared to the wild type sequence). A variant PhrE protein can contain an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99 identical to SEQ ID NO:9.

As indicated above, many different proteins find use as a POI in the Gram positive cell (i.e., the protein whose expression is increased in the genetically altered cell). The POI can be a homologous protein or a heterologous protein, and may be a wild-type protein, a natural variant or a recombinant variant. In certain embodiments, the POI is an enzyme, where in certainembodiments, the enzyme is selected from acetyl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, rhamno-galacturonases, ribonucleases, transferases, transport proteins, transglutaminases, xylanases, hexose oxidases, and combinations thereof

In certain other embodiments, the POI is a protease, wherein the protese may be a subtilisin, e.g., a subtilisin selected from subtilisin 168, subtilisin BPN', subtilisin Carlsberg, subtilisin DY, subtilisin 147, subtilisin 309, and variants thereof. In certain embododimetns, the POI is a fluorescent protein, e.g., a green fluorescent protein (GFP).

In certain embodiments, the methods and compositions thereof further comprise recovering the protein of interest. Because the level of expression/production of the protein of interest is increased in the genetically altered Gram positive (daughter) cell (as compared to the unaltered parental cell), the amount of the POI recovered is increased relative to the corresponding Gram positive (parental) cell, when cultured under essentiall the same culture conditions (and at the same scale). There are various assays known to those of ordinary skill in the art for detecting and measuring the expression level/production of intracellularly and extracellularly expressed polypeptides. Such assays are determined by the user of the present invention and may depend on the identity and/or activity (e.g., enzymatic activity) of the POI. For example, for assaythe proteases, there are assays based on the release of acid-soluble peptides from casein or hemoglobin measured as absorbance at 280 nm or colorimetrically using the Folin method (See e.g., Bergmeyer et al., “Methods of Enzymatic Analysis” vol. 5, Peptidases, Proteinases and their Inhibitors, Verlag Chemie, Weinheim [1984]). Other assays involve the solubilization of chromogenic substrates (See e.g., Ward, “Proteinases,” in Fogarty (ed.)., Microbial Enzymes and Biotechnology, Applied Science, London, [1983], pp 251-317). Other examples of assays include succinyl-Ala-Ala-Pro-Phe-para nitroanilide assay (SAAPFpNA) and the 2,4,6-trinitrobenzene sulfonate sodium salt assay (TNBS assay). Numerous additional references known to those in the art provide suitable methods (See e.g., Wells et al., Nucleic Acids Res. 11:7911-7925 [1983]; Christianson et al., Anal. Biochem., 223:119 -129 [1994]; and Hsia et al., Anal Biochem., 242:221-227 [1999]).

Also as indicated above, means for determining the levels of secretion of a POI in a host cell and detecting expressed proteins include the use of immunoassays with either polyclonal or monoclonal antibodies specific for the protein of interest. Examples include enzyme-linked immunosorbent assay (ELISA), radioimmunoassay (RIA), fluorescence immunoassay (FIA), and fluorescent activated cell sorting (FACS). However, other methods are known to those in the art and find use in assessing the protein of interest (See e.g., Hampton et al., Serological Methods, A Laboratory Manual, APS Press, St. Paul, Minn. [1990]; and Maddox et al., J. Exp. Med., 158:1211 [1983]). As known in the art, the altered Bacillus cells produced using the present invention are maintained and grown under conditions suitable for the expression and recovery of a POI from cell culture (See e.g., Hardwood and Cutting (eds.) Molecular Biological Methods for Bacillus, John Wiley & Sons [1990]). It is further noted that a genetically altered cell as described herein may express more than one POI, including two or more, three or more, four or more, five or more, six or more, seven or more, eight or more, nine or more, ten or more, etc. In some embodiments, increased expression of proteins in the bacterial secretome is desired, which includes numerous different proteins that are secretred from the cell.

Aspects of the present invention include methods for obtaining an altered Gram positive bacterial cell with improved protein production capability. In general, the methods include genetically altering a parental Gram positive cell to result in a genetically altered daughter Gram positive cell, wherein the expression of one or more genes that activate the phosphorelay system (as defined above).

In certain embodiments, the method includes introducing a polynucleotide sequence into a parental Gram positive bacterial cell that, when integrated into the chromosome or sustained as an episomal genetic element, results in a genetically altered Gram positive cell in which the expression level of one or more genes that activates the phosphorelay system.

Various methods are known for the transformation of Bacillus species to alter the chromosome, or to maintain an episomal genetic element in the Bacillus cell, using polynucldotide vectors (e.g., plasmid constructs) which are well known to one of skill in the art. Suitable methods for introducing polynucleotide sequences into Bacillus cells are found in, e.g., Ferrari et al, “Genetics,” in Harwood et al. (ed.), Bacillus, Plenum Publishing Corp. [1989], pages 57-72; See also, Saunders et al., J. Bacteriol., 157:718-726 [1984]; Hoch et al., J. Bacteriol., 93:1925 -1937 [1967]; Mann et al., Current Microbiol., 13:131-135 [1986]; and Holubova, Folia Microbiol., 30:97 [1985]; for B. subtilis, Chang et al., Mol. Gen. Genet., 168:11-115 [1979]; for B. megaterium, Vorobjeva et al., FEMS Microbiol. Lett., 7:261-263 [1980]; for B amyloliquefaciens, Smith et al., Appl. Env. Microbiol., 51:634 (1986); for B. thuringiensis, Fisher et al., Arch. Microbiol., 139:213-217 [1981]; and for B. sphaericus, McDonald, J. Gen. Microbiol.,130:203 [1984]. Indeed, such methods as transformation including protoplast transformation and congression, transduction, and protoplast fusion are known and suited for use in the present invention. Methods of transformation are particularly to introduce a DNA construct provided by the present invention into a host cell

In addition, introduction of a DNA construct into the host cell includes physical and chemical methods known in the art to introduce DNA into a host cell without insertion of the targeting DNA construct into a plasmid or vector. Such methods include, but are not limited to calcium chloride precipitation, electroporation, naked DNA, liposomes and the like. In additional embodiments, DNA constructs can be co-transformed with a plasmid, without being inserted into the plasmid.

In embodiments in which selectable marker genes are used to select for stble transformants, it may be desireable to delete the selective marker from the genetically altered Gram positive strain using any convenient method, with numerous methods being known in the art (See, Stahl et al., J. Bacteriol., 158:411-418 [1984]; and Palmeros et al., Gene 247:255 -264 [2000]).

In some embodiments, two or more DNA constructs (i.e., DNA constructs that each are designed to genetically alter a host cell) are introduced into a parental Gram positive cell, resulting in the introduction of two or more genetic alterations in the cell, e.g., alterations at two or more chromosomal regions. In some embodiments, these regions are contiguous, (e.g., two regions within a single operon), while in other embodiments, the regions are separated. In some embodiments, one or more of the genetic alterations are by addition of an episomal genetic element.

In some embodiments, host cells are transformed with one or more DNA constructs according to the present invention to produce an altered Bacillus strain wherein two or more genes have been inactivated in the host cell. In some embodiments, two or more genes are deleted from the host cell chromosome. In alternative embodiments, two or more genes are inactivated by insertion of a DNA construct. In some embodiments, the inactivated genes are contiguous (whether inactivated by deletion and/or insertion), while in other embodiments, they are not contiguous genes.

Once a genetically altered host cell is produced, it can be cultured under conditions such that the protein of interest is expressed, where in certain embodiments the POI is recovered.

In some embodiments, the present invention includes a DNA construct comprising an incoming sequence that, when stably incorporated into the host cell, genetically alters the cell such that expression of one or more genes that activates the phosphorelay system that induces the expression of sporulation-initiating genesis reduced (as described in detail above). In some embodiments, the DNA construct is assembled in vitro, followed by direct cloning of the construct into a competent Gram positive (e.g., Bacillus) host such that the DNA construct becomes integrated into the host cell chromosome. For example, PCR fusion and/or ligation can be employed to assemble a DNA construct in vitro. In some embodiments, the DNA construct is a non-plasmid construct, while in other embodiments it is incorporated into a vector (e.g., a plasmid). In some embodiments, circular plasmids are used. In embodiments, circular plasmids are designed to use an appropriate restriction enzyme (i.e., one that does not disrupt the DNA construct). Thus, linear plasmids find use in the present invention. However, other methods are suitable for use in the present invention, as known to those in the art (See e.g., Perego, “Integrational Vectors for Genetic Manipulation in Bacillus subtilis,” in (Sonenshein et al. (eds.), Bacillus subtilis and Other Gram-Positive Bacteria, American Society for Microbiology, Washington, D.C. [1993]).

In certain embodiments, the DNA targeting vector is designed to delete (or allow for the deletion of) all or part of the kinA gene, the phrA gene, or the phrE gene. In certain embodiments, multiple DNA constructs are employed, either simultaneously or sequentially, to delete any two or three of the kinA gene, the phrA gene, and the phrE gene. In certain embodiments, the DNA targeting vector includes a selective marker. In some embodiments, the selective marker is located between two loxP sites (See, Kuhn and Torres, Meth. Mol. Biol.,180:175-204 [2002]), and the antimicrobial gene is then deleted by the action of Cre protein.

Aspects of the present invention include a method for enhancing expression of a POI in a Gram positive bacterial cell that includes transforming a parental Gram positive bacterial cell with the DNA construct or vector described above (i.e., one that includes an incoming sequence that, when stably incorporated into the host cell, genetically alters the cell such that expression of one or more genes of the phosphorelay pathway is reduced), allowing homologous recombination of the vector and the corresponding region in thegene of interest of the parental Gram positive bacterial cell to produce an altered Gram positive bacterial cell; and growing the altered Gram positive bacterial cell under conditions suitable for the expression of the POI, where the production of the POI is increased in the altered Gram positive bacterial (daughter) cell as compared to the Gram positive bacterial (parental) cell. Examples of the Gram positive cells, mutations and other features that find use in this aspect of the invention are described in detail above.

Whether the DNA construct is incorporated into a vector or used without the presence of plasmid DNA, it is used to transform microorganisms. It is contemplated that any suitable method for transformation will find use with the present invention. In certain embodiments, at least one copy of the DNA construct is integrated into the host Bacillus chromosome. In some embodiments, one or more DNA constructs of the invention are used to transform host cells.

The manner and method of carrying out the present invention may be more fully understood by those of skill in the art by reference to the following examples, which examples are not intended in any manner to limit the scope of the present invention or of the claims directed thereto.

### EXAMPLES

The following Examples are provided in order to demonstrate and further illustrate certain embodiments and aspects of the present invention and are not to be construed as limiting the scope thereof.

In the experimental disclosure which follows, certain of the following abbreviations apply: ° C. (degrees Centigrade); rpm (revolutions per minute); μg (micrograms); mg (milligrams); μl (microliters); ml (milliliters); mM (millimolar); μM (micromolar); sec (seconds); min(s) (minute/minutes); hr(s) (hour/hours); OD₂₈₀ (optical density at 280 nm); OD₆₀₀ (optical density at 600 nm); PCR (polymerase chain reaction); RT-PCR (reverse transcription PCR); SDS (sodium dodecyl sulfate).

### Example 1

### Increased Protein Expression in Bacillus by Deletion in the kinA Gene

A. Deletion of kinA Locus in Bacillus Subtilis

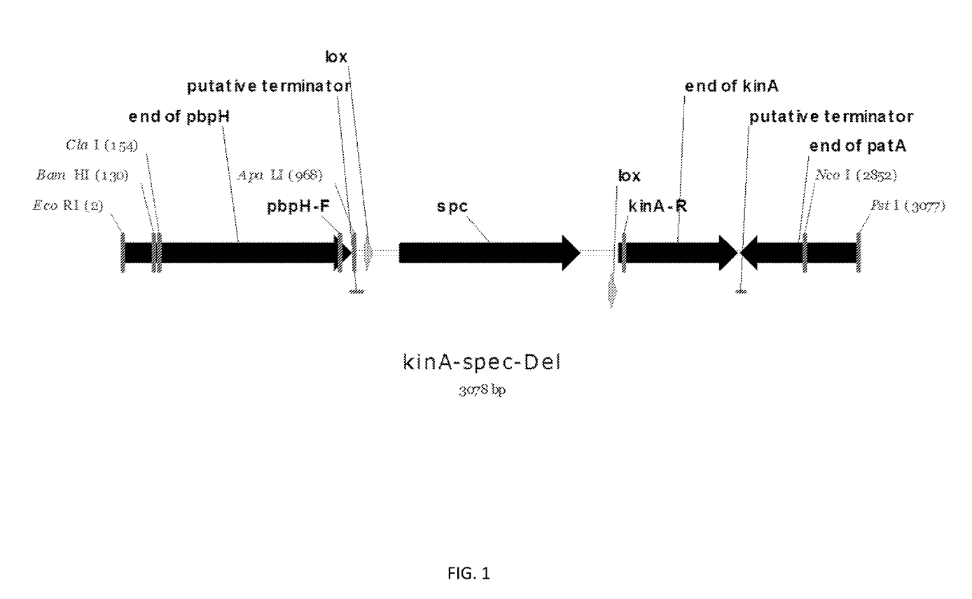

A deletion in kinA was introduced into parental Bacillus subtilis cells by homologous recombination using a kinA deletion cassette (FIG. 1). The deletion was confirmed by PCR and sequencing of the kinA locus. The resultant daughter cells was denoted by ΔkinA and the unaltered Bacillus subtilis cells,referred to herein as parentalcells. SEQ ID NO: 1 shows the wildtype sequence of the kinA gene, and SEQ ID NO: 2 shows the KinA protein sequence.

B. Amylase Expression in the kinA Deleted Strain

An amylase expression construct which drives the expression of AmyE from the aprE promoter, and which includes a chloramphenicol acetyltransferase resistance (catR) marker gene (herein, “PaprE-amyE catR”) was introduced into the aprE locus of the of both the ΔkinA (daughter cells) and the unaltered parental cells. The mature AmyE protein sequence shown in SEQ ID NO: 3.

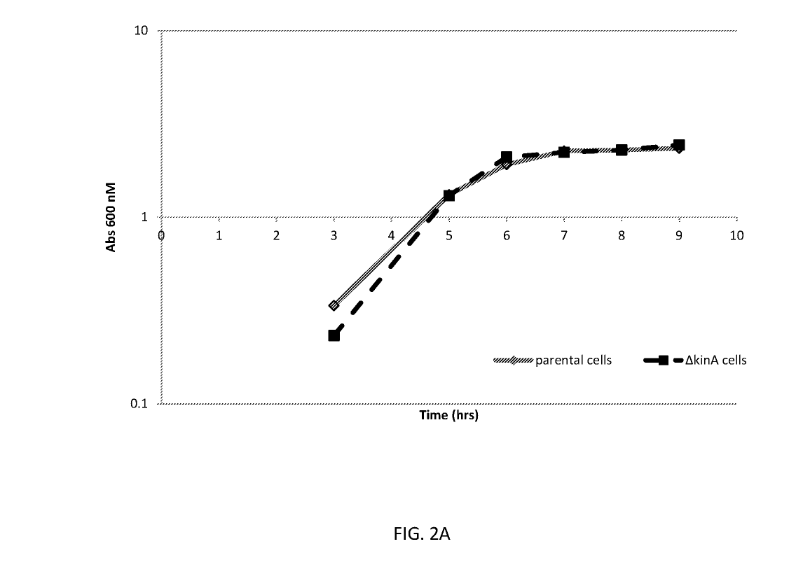

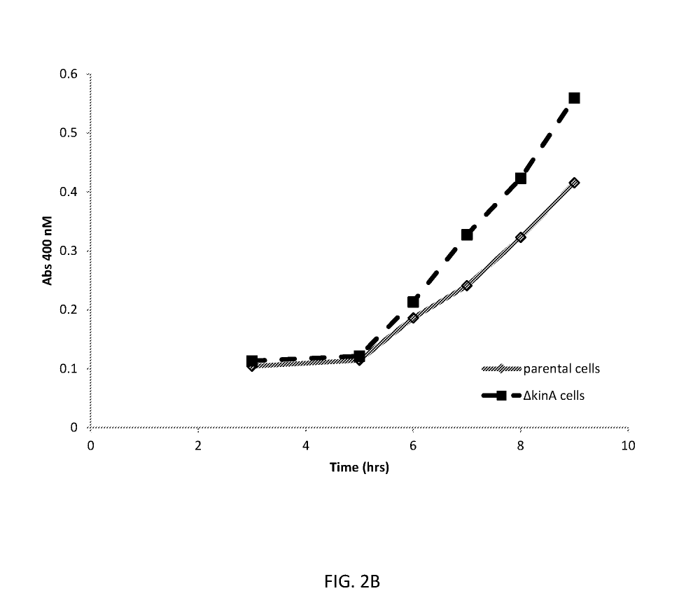

The cells were amplified on Luria agar plates containing 25 μg/ml of chloramphenicol. The ΔkinA (daughter) cells and the parental cells were grown overnight in 5 mL of Luria broth medium. One (1) ml of pre-culture was used to inoculate 25 ml of Luria broth medium in shake flasks at 37° C., 250 rpm to test the expression of the AmyE amylase protein. Cell densities were measured at 600 nm at hourly intervals using a SpectraMax spectrophotometer (Molecular Devices, Downington, PA, USA). The absorbance at 600 nm was plotted as a function of time and the results are shown in FIG. 2A. For example, FIG. 2A shows that the cell growth of the parental cells and the ΔkinA (daughter) cells is equivalent, indicating that the deletion of the kinA gene in the (daughter) cells does not affect the cell growth.

The AmyE amylase activity of whole broth was measured using the Ceralpha reagent (Megazyme, Wicklow, Ireland.). The Ceralpha reagent mix from the Ceralpha HR kit was initially dissolved in 10 ml of MilliQ water followed by the addition of 30 ml of 50 mM malate buffer, pH 5.6. The culture supernatants were diluted 40× in MilliQ water and 5 μl of diluted sample was added to 55 μL of diluted working substrate solution. The MTP plate was incubated for 4 minutes at room temperature after shaking. The reaction was quenched by adding 70 μl of 200 mM borate buffer pH 10.2 (stop solution). The absorbance of the solution was measured at 400 nm using a SpectraMax spectrophotometer (Molecular Devices, Downington, Pa., USA). The absorbance at 400 nm was plotted as a function of time and the results are shown in FIG. 2B. The graph in FIG. 2B shows increased AmyE production starting at 6 hours of growth in the altered (ΔkinA; daughter) cells. Given that cell growth was not affected in the altered (ΔkinA; daughter) cells (as shown in FIG. 2A), the increase in AmyE production in altered (ΔkinA; daughter) Bacillus cells relative to the unaltered (parenta) Bacillus cells (grown under the same culture conditions) is not due to an increase in the number of cells in the culture, but rather due to increased expression levels in the cells themselves (i.e., on a cell-by-cell basis).

C. Protease (FNA) Expression in the kinA Deleted Strain

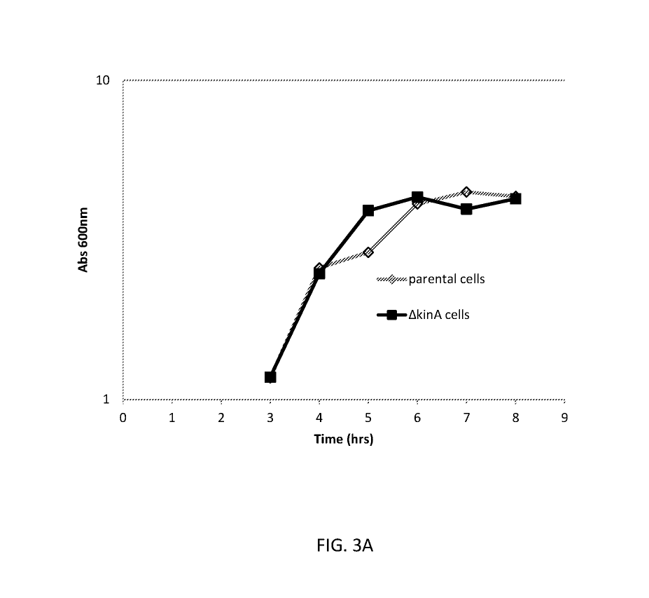

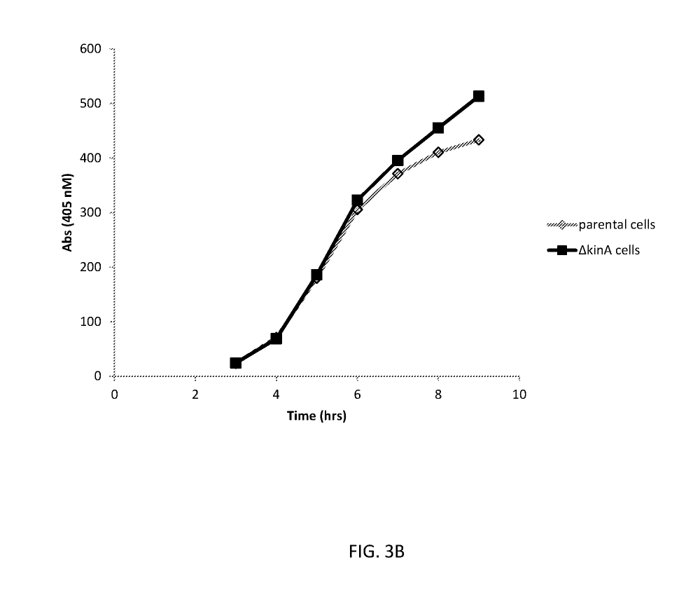

The effect of the kinA deletion (ΔkinA) on expression of FNA protease (subtilisin BPN' containing the Y217L substitution; SEQ ID NO: 4) was tested in Bacillus subtilis cells comprising an FNA expression cassette (herein, “PaprE-FNA-catR”). The kinA gene in the altered B. subtilis (daughter) cells was deleted by transformation of the strain with the construct shown in FIG. 1. The spectinomycin resistant colonies carrying the deletion of kinA were amplified on LA plates containing 25 μg/ml chloramphenicol. The parental B. subtilis cells and the ΔkinA knockout daughter cells were grown overnight in 5 mL of Luria broth medium. One (1) ml of pre-culture was used to inoculate 25 ml of 2×NB (2× Nutrient Broth, 1×SNB salts, described in PCT International Publication No WO2010/14483) in Thompson flasks at 250 rpm to test protease expression. Cell densities of whole broth were diluted 20× and measured at 600 nm at hourly intervals using a SpectraMax spectrophotometer (Molecular Devices, Downington, Pa., USA). The absorbance at 600 nm was plotted as a function of time and the results are presented in FIG. 3A,which show that the cell growth of the FNA expressing B. subtilis parental cells and the FNA expressing B. subtilis daughter cells (i.e., ΔkinA) is equivalent, indicating that the kinA deletion in the daughter cells does not affect the cell growth.

FNA Protease expression was monitored using N-suc-AAPF-pNA substrate (from Sigma Chemical Co.) as described in PCT International Publication No. WO 2010/144283. Briefly, whole broth was diluted 40× in the assay buffer (100 mM Tris, 0.005% Tween 80, pH 8.6) and 10 μl of the diluted samples were arrayed in microtiter plates. The AAPF stock was diluted in the assay buffer (100× dilution of 100 mg/ml AAPF stock in DMSO) and 190 μl of this solution were added to the microtiter plates and the absorbance of the solution was measured at 405 nm using a SpectraMax spectrophotometer (Molecular Devices, Downington, Pa., USA). The absorbance at 405 nm was plotted as a function of time and the results are presented in FIG. 3B, which shows that FNA production is increased in daughter Bacillus cell cultures comprising the the ΔkinA deletion as compared to cultures of the parental Bacillus cells grown under the same culture conditions. Given that cell growth was not affected in the altered (ΔkinA) Bacillus daughter cells (as shown in FIG. 3A), the increase in FNA production in Bacillus (ΔkinA) daughter cells relative to the unaltered parental Bacillus cells (grown under the same culture conditions) is not due to an increase in the number of cells in the culture, but rather is due to increased expression levels in the altered cells themselves (i.e., on a cell-by-cell basis).

D. Green Fluorescent Protein (GFP) Expression in the kinA Deleted Strain

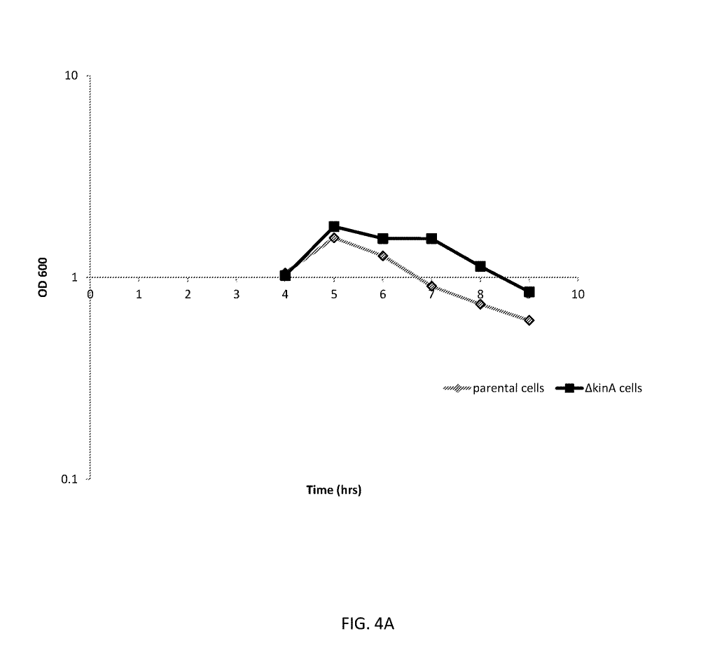

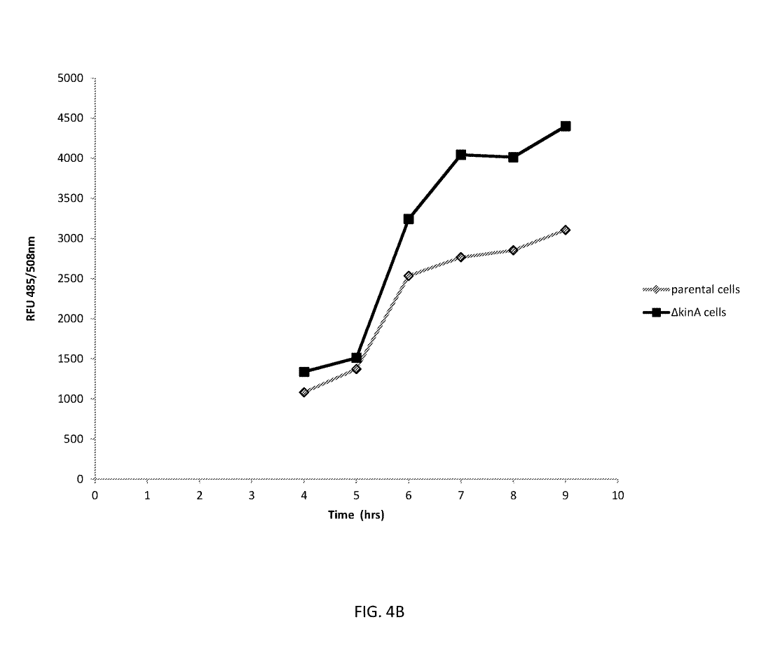

To test the effect of the kinA deletion on expression of other proteins, a GFP expression cassette (herein, “PaprE-GFP catR”), under the control of the aprE promoter, and further comprising a chloramphenicol acetyltransrefase resistance marker (SEQ ID NO: 5 shows the amino acid sequence of GFP), was introduced in the aprE locus of the unaltered B. subtilis parental cells and the altered (ΔkinA) B. subtilis daughter cells. Transformants were selected on Luria agar plates containing 5 μg/ml of chloramphenicol. The altered B. subtilis (ΔkinA) daughter cells expressing GFP and the unaltered B. subtilis parental cells expressing GFP were grown overnight in 5 mL of Luria broth. One (1) ml of pre-culture was used to inoculate 25 ml of 2×NB medium (2× nutrient broth, 1×SNB salts) in shake flasks at 37° C., 250 rpm to test the expression of green fluorescent protein (GFP). Cell densities of whole broth diluted 20× were measured at 600 nm at hourly intervals using a SpectraMax spectrophotometer (Molecular Devices, Downington, Pa., USA). The absorbance at 600 nm was plotted as a function of time and the results are presented in FIG. 4A, which shows that the cell growth of the GFP expressing B. subtilis parental cells is reduced as compared to the GFP expressing B. subtilis (ΔkinA) daughter cells, indicating that the kinA deletion in these GFP expressing cells positively affects cell growth.

To measure GFP expression, 100 μl of culture was transferred to a 96 well microtiter plate and GFP expression was measured in a fluorescent plate reader using an excitation wavelength of 485 nm, an emission wavelength of 508 nm with a 495 nm emission cutoff filter. The relative fluorescence units (RFU) at 485/508 nm were plotted as a function of time and the results are shown in FIG. 4B. The graph shows an increase of GFP production from 6 hours of growth due to the kinA deletion. The level of increased GFP expression in the altered B. subtilis (ΔkinA) daughter cells as compared to the unaltered B. subtilis parental cells exceeds what would be expected merely from the improvement in cell viability seen in FIG. 4A.

### Example 2

Increased Protein Expression in Bacillus by Deletion of the phrA and phrE Genes

A. Deletion of phrA Locus in Bacillus subtilis

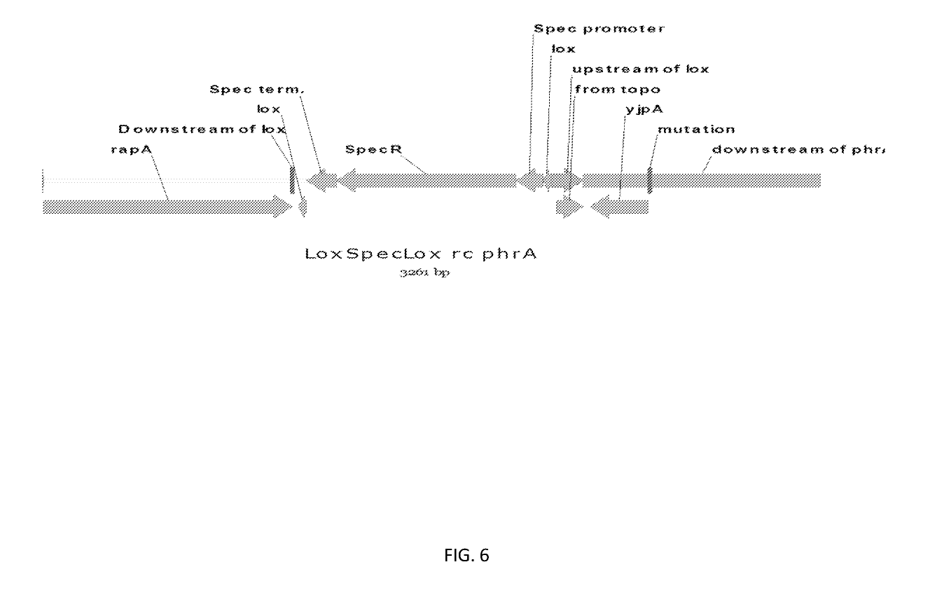

A deletion in the phrA gene was introduced into parental Bacillus subtilis cells by homologous recombination of a deletion cassette presented shcemiatically in FIG. 6. The phrA deletion (ΔphrA) was confirmed by PCR and sequencing of the phrA locus. The spectinomycin marker (specR) was removed using a plasmid encoded Cre recombinase.

B. Deletion of phrE Locus in Altered (ΔphrA) Bacillus cells

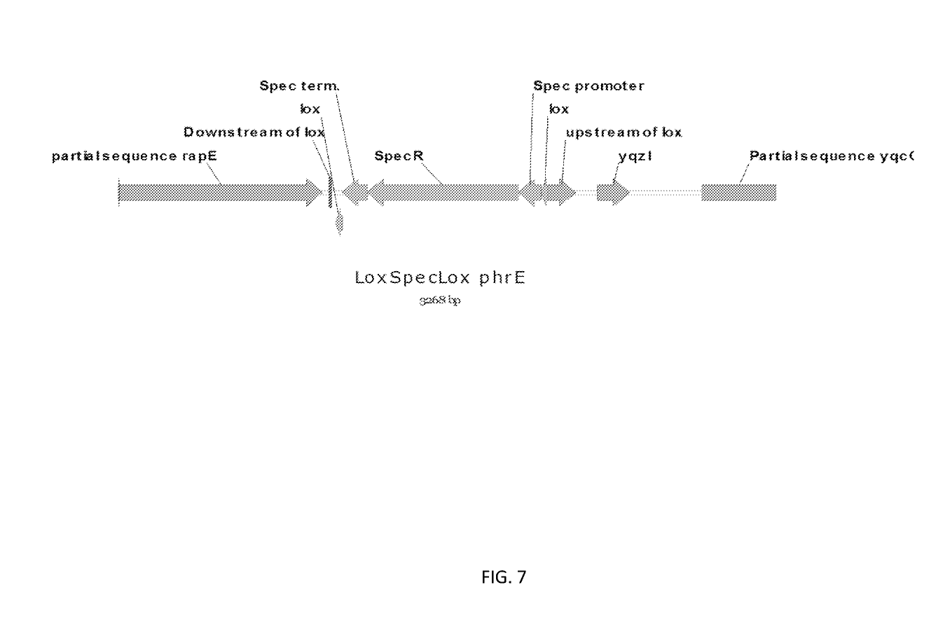

The phrE gene was also deleted in the Example 2.A described altered (ΔAphrA) B. subtilis daughter cells by homologous recombination of a deletion cassette presented schematically in FIG. 7). The phrE deletion was confirmed by PCR and sequencing of the phrE locus.

C. Protein Expression in the Altered (ΔphrA/ΔphrE) Bacillus cells

The expression cassettes previously described in Example 1 (i.e., the “PaprE-FNA catR” cassette, the “PaprE-GFP catR” cassette or the “PaprE-amyE catR” cassette,were introduced into the chromosome of the unaltered (parental) B. subtilis cells and the altered (ΔphrA/ΔphrE) B. subtilis cells. Strain selection, cell growth, and enzyme assays were performed as described in Example 1. The B. subtilis cells comprising the “PaprE-FNA catR” cassette were selected on chloramphenicol 25 ppm plates. The B. subtilis cells comprising the “PaprE-GFP catR” cassette or the “PaprE-AmyE catR” cassette were selected on chloramphenicol 5 ppm plates. Cell densities and protein expression were measured as described in Example 1.

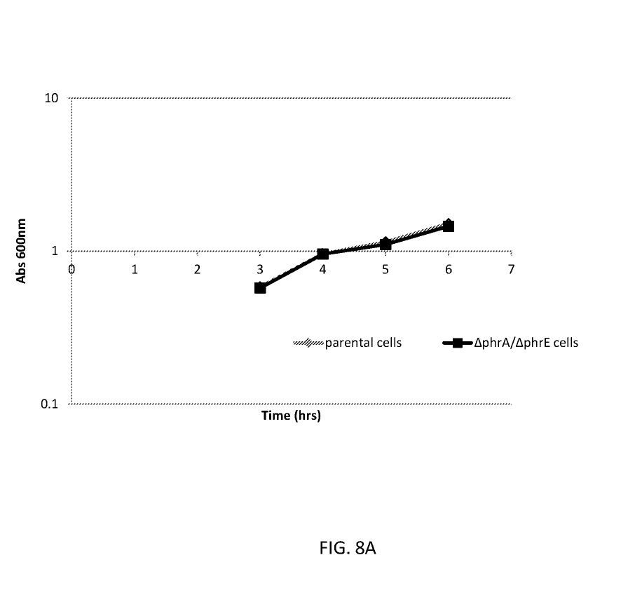

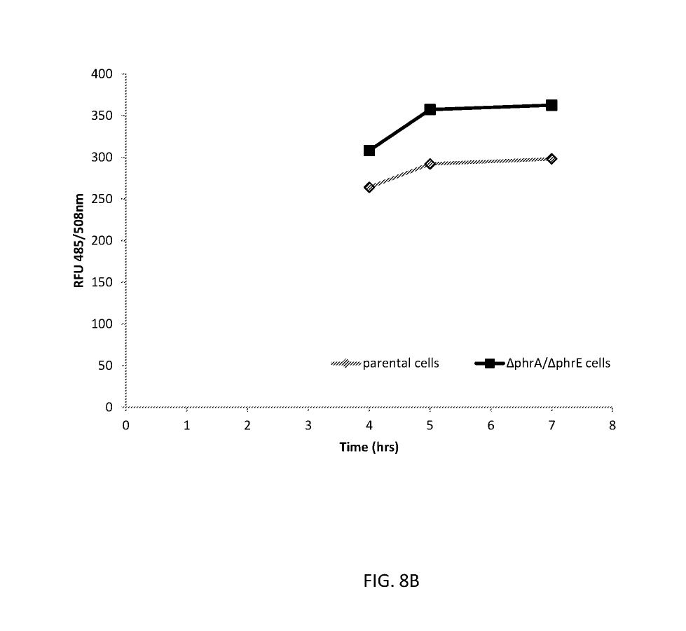

FIG. 8A shows that the cell growth of the GFP expressing parental B. subtilis cells and the GFP expressing daughter (ΔphrA/ΔphrE) B. subtilis cells is equivalent, indicating that the phrA-phrE deletion in the B. subtilis (daughter) cells does not affect the cell growth. FIG. 8B shows an increase in GFP production from 4 hours of growth as a result of the phrA-phrE deletion.

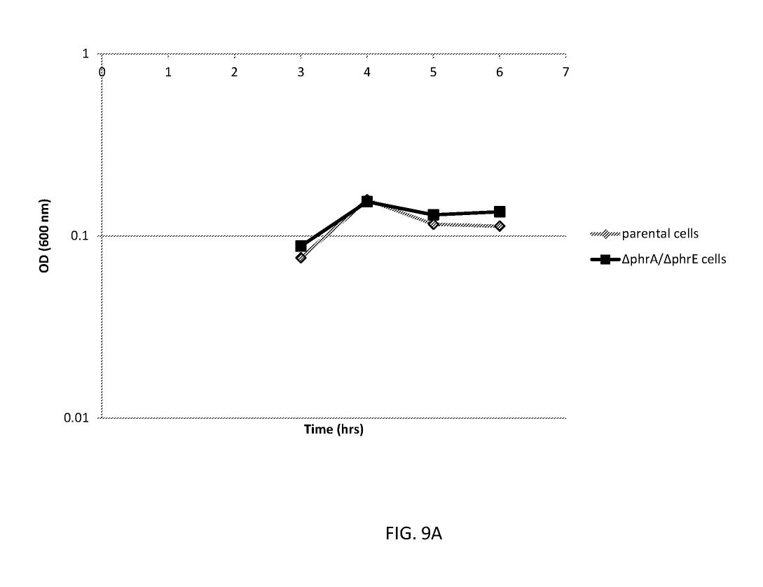

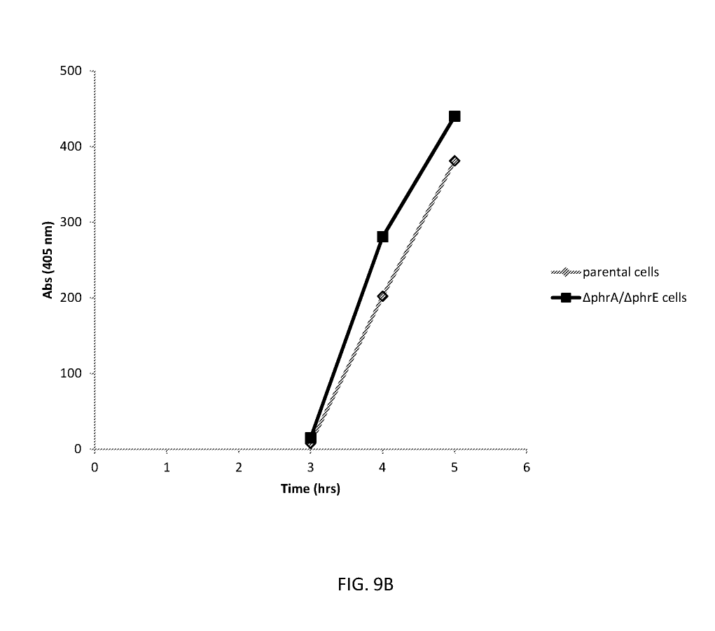

FIG. 9A shows that the cell growth of the FNA expressing parental B. subtilis cells and the FNA expressing daughter (ΔphrA/ΔphrE) B. subtilis cells is equivalent, indicating that the phrA-phrE deletion in the B. subtilis (daughter) cells does not affect the cell growth. FIG. 9B shows an increase of FNA production from 4 hours of growth as a result of the phrA-phrE deletion.

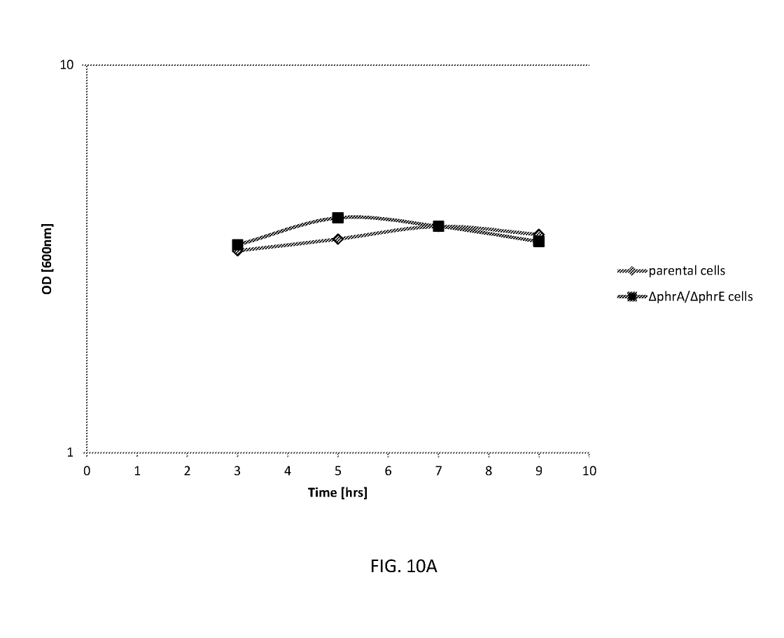

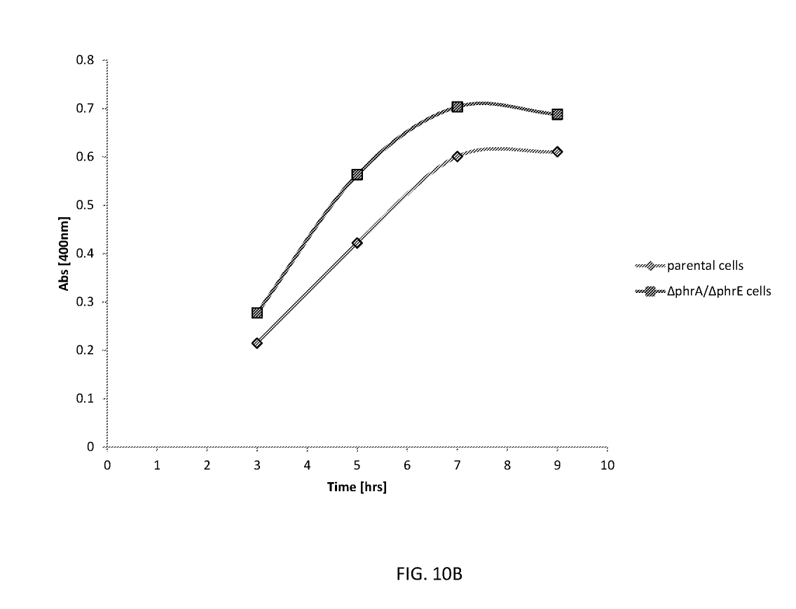

FIG. 10A shows that the cell growth of the AmyE expressing parental B. subtilis cells and the AmyE expressing daughter (ΔphrA/ΔphrE) B. subtilis cells is equivalent, indicating that the phrA-phrE deletion in the B. subtilis (daughter) cells does not affect the cell growth. FIG. 10B shows an increase in AmyE production as a result of the phrA-phrE deletion.

Although the foregoing compositions and methods have been described in some detail by way of illustration and example for purposes of clarity of understanding, it is readily apparent to those of ordinary skill in the art in light of the teachings herein that certain changes and modifications may be made thereto without departing from the spirit or scope of the appended claims.

Accordingly, the preceding merely illustrates the principles of the present compositions and methods. It will be appreciated that those skilled in the art will be able to devise various arrangements which, although not explicitly described or shown herein, embody the principles of the present compositions and methods and are included within its spirit and scope. Furthermore, all examples and conditional language recited herein are principally intended to aid the reader in understanding the principles of the present compositions and methods and the concepts contributed by the inventors to furthering the art, and are to be construed as being without limitation to such specifically recited examples and conditions. Moreover, all statements herein reciting principles, aspects, and embodiments of the present compositions and methods as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present compositions and methods, therefore, is not intended to be limited to the exemplary embodiments shown and described herein.

### SEQUENCES

kinA wildtype coding sequence
SEQ ID NO: 1
GTGGAACAGGATACGCAGCATGTTAAACCACTTCAAACAAAAACCGATATTCATGCAGTCTT
GGCCTCTAATGGACGCATCATTTATATATCTGCCAACTCCAAACTGCATTTGGGCTATCTCC
AAGGAGAGATGATCGGATCATTCCTCAAAACGTTTCTGCATGAGGAAGACCAATTTTTGGTT
GAAAGCTATTTTTATAATGAACATCATCTGATGCCGTGCACCTTTCGTTTTATTAAAAAAGA
TCATACGATTGTGTGGGTGGAGGCTGCGGTAGAAATTGTTACGACAAGAGCTGAGCGGACAG
AACGGGAAATCATTTTGAAAATGAAGGTTCTTGAAGAAGAAACAGGCCATCAATCCCTAAAC
TGCGAAAAACATGAAATCGAACCTGCAAGCCCGGAATCGACTACATATATAACGGATGATTA
TGAACGGTTGGTTGAAAATCTCCCGAGTCCGCTATGCATCAGTGTCAAAGGCAAGATCGTCT
ATGTAAACAGCGCGATGCTTTCAATGCTGGGAGCCAAAAGCAAGGATGCTATTATTGGTAAA
TCGTCCTATGAATTTATTGAAGAAGAATATCATGATATCGTGAAAAACAGGATTATACGAAT
GCAAAAAGGAATGGAAGTCGGAATGATTGAACAGACGTGGAAAAGGCTTGATGGCACACCTG
TTCATTTAGAAGTGAAAGCATCCCCGACCGTCTACAAAAACCAGCAGGCTGAGCTGCTGCTG
CTGATCGATATCTCTTCAAGGAAAAAATTCCAAACCATCCTGCAAAAAAGCCGTGAACGATA
TCAGCTGCTGATTCAAAATTCCATTGATACCATTGCGGTGATTCACAATGGAAAATGGGTAT
TTATGAATGAATCGGGAATTTCCCTGTTTGAAGCGGCTACATATGAGGATTTAATTGGCAAA
AACATATACGATCAGCTGCATCCTTGCGATCACGAGGATGTAAAAGAGAGAATCCAAAACAT
TGCCGAGCAAAAAACAGAATCTGAAATTGTCAAGCAATCCTGGTTCACCTTTCAGAACAGGG
TCATCTATACGGAGATGGTCTGCATTCCGACGACCTTTTTTGGTGAAGCGGCCGTCCAGGTC
ATTCTTCGGGACATCTCAGAGAGAAAACAAACAGAAGAATTGATGCTGAAATCGGAAAAATT
ATCAATCGCAGGGCAGCTCGCGGCGGGAATCGCCCATGAGATCCGCAACCCTCTTACAGCGA
TCAAAGGATTTTTACAGCTGATGAAACCGACAATGGAAGGCAACGAACATTACTTTGATATT
GTGTTTTCTGAACTCAGCCGTATCGAATTAATACTCAGTGAACTGCTCATGCTGGCGAAACC
TCAGCAAAATGCTGTCAAAGAATATTTGAACTTGAAAAAATTAATTGGTGAGGTTTCAGCCC
TGTTAGAAACGCAGGCGAATTTAAATGGCATTTTTATCAGAACAAGTTATGAAAAAGACAGC
ATTTATATAAACGGGGATCAAAACCAATTAAAGCAGGTATTCATTAATTTAATCAAAAATGC
AGTTGAATCAATGCCTGATGGGGGAACAGTAGACATTATCATAACCGAAGATGAGCATTCTG
TTCATGTTACTGTCAAAGACGAAGGGGAAGGTATACCTGAAAAGGTACTAAACCGGATTGGA
GAGCCATTTTTAACAACAAAAGAAAAAGGTACGGGGCTTGGATTAATGGTGACATTTAATAT
CATTGAAAACCATCAGGGAGTTATACATGTGGACAGCCATCCTGAAAAAGGCACAGCGTTTA
AAATTTCATTTCCAAAAAAATAA
KinA protein sequence
SEQ ID NO: 2
MEQDTQHVKPLQTKTDIHAVLASNGRIIYISANSKLHLGYLQGEMIGSFLKTFLHEEDQFLV
ESYFYNEHHLMPCTFRFIKKDHTIVWVEAAVEIVTTRAERTEREIILKMKVLEEETGHQSLN
CEKHEIEPASPESTTYITDDYERLVENLPSPLCISVKGKIVYVNSAMLSMLGAKSKDAIIGK
SSYEFIEEEYHDIVKNRIIRMQKGMEVGMIEQTWKRLDGTPVHLEVKASPTVYKNQQAELLL
LIDISSRKKFQTILQKSRERYQLLIQNSIDTIAVIHNGKWVFMNESGISLFEAATYEDLIGK
NIYDQLHPCDHEDVKERIQNIAEQKTESEIVKQSWFTFQNRVIYTEMVCIPTTFFGEAAVQV
ILRDISERKQTEELMLKSEKLSIAGQLAAGIAHEIRNPLTAIKGFLQLMKPTMEGNEHYFDI
VFSELSRIELILSELLMLAKPQQNAVKEYLNLKKLIGEVSALLETQANLNGIFIRTSYEKDS
IYINGDQNQLKQVFINLIKNAVESMPDGGTVDIIITEDEHSVHVTVKDEGEGIPEKVLNRIG
EPFLTTKEKGTGLMVTFNIIENHQGVIHVDSHPEKGTAFKISFPKK
AmyE protein sequence
SEQ ID NO: 3
LTAPSIKSGTILHAWNWSFNTLKHNMKDIHDAGYTAIQTSPINQVKEGNQGDKSMSNWYWLY
QPTSYQIGNRYLGTEQEFKEMCAAAEEYGIKVIVDAVINHTTSDYAAISNEVKSIPNWTHGN
TQIKNWSDRWDVTQNSLLGLYDWNTQNTQVQSYLKRFLDRALNDGADGFRFDAAKHIELPDD
GSYGSQFWPNITNTSAEFQYGEILQDSASRDAAYANYMDVTASNYGHSIRSALKNRNLGVSN
ISHYASDVSADKLVTWVESHDTYANDDEESTWMSDDDIRLGWAVIASRSGSTPLFFSRPEGG
GNGVRFPGKSQIGDRGSALFEDQAITAVNRFHNVMAGQPEELSNPNGNNQIFMNQRGSHGVV
LANAGSSSVSINTATKLPDGRYDNKAGAGSFQVDGKLTGTINARSVAVLYPD
FNA protein sequence
SEQ ID NO: 4
AGKSNGEKKYIVGFKQTMSTMSAAKKKDVISEKGGKVQKQFKYVDAASATLNEKAVKELKKD
PSVAYVEEDHVAHAYAQSVPYGVSQIKAPALHSQGYTGSNVKVAVIDSGIDSSHPDLKVAGG
ASMVPSETNPFQDNNSHGTHVAGTVAALNNSIGVLGVAPSASLYAVKVLGADGSGQYSWIIN
GIEWAIANNMDVINMSLGGPSGSAALKAAVDKAVASGVVVVAAAGNEGTSGSSSTVGYPGKY
PSVIAVGAVDSSNQRASFSSVGPELDVMAPGVSIQSTLPGNKYGALNGTSMASPHVAGAAAL
ILSKHPNWTNTQVRSSLENTTTKLGDSFYYGKGLINVQAAAQ
GFP protein sequence
SEQ ID NO: 5
VNRNVLKNTGLKEIMSAKASVEGIVNNHVFSMEGFGKGNVLFGNQLMQIRVTKGGPLPFAFD
IVSIAFQYGNRTFTKYPDDIADYFVQSFPAGFFYERNLRFEDGAIVDIRSDISLEDDKFHYK
VEYRGNGFPSNGPVMQKAILGMEPSFEVVYMNSGVLVGEVDLVYKLESGNYYSCHMKTFYRS
KGGVKEFPEYHFIHHRLEKTYVEEGSFVEQHETAIAQLTTIGKPLGSLHEWV
phrA wildtype coding sequence
SEQ ID NO: 6
ATGAAATCTAAATGGATGTCAGGTTTGTTGCTCGTTGCGGTCGGGTTCAGCTTTACTCAGGT
GATGGTTCATGCAGGTGAAACAGCAAACACAGAAGGGAAAACATTTCATATTGCGGCACGCA
ATCAAACAtGA
phrA protein sequence
SEQ ID NO: 7
MKSKWMSGLLLVAVGFSFTQVMVHAGETANTEGKTFHIAARNQT
phrE wildtype coding sequence
SEQ ID NO: 8
ATGAAATCTAAATTGTTTATCAGTTTATCCGCCGTTTTAATTGGACTTGCCTTTTTCGGATC
TATGTATAATGGCGAAATGAAGGAAGCATCCCGGAATGTAACTCTCGCACCTACTCATGAAT
TCCTTGTTTAA
phrE protein sequence
SEQ ID NO: 9
MKSKLFISLSAVLIGLAFFGSMYNGEMKEASRNVTLAPTHEFLV

1. A method for increasing expression of a protein of interest (POI) in a Gram positive bacterial cell comprising:
(a) obtaining an altered Gram positive bacterial cell producing a POI, wherein said altered Gram positive bacterial cell comprises at least one genetic alteration that reduces expression of a kinA gene, a phrA gene or a phrE gene; and
(b) culturing said altered Gram positive bacterial cell under conditions such that said POI is expressed,
wherein the increased expression of the POI is relative to the expression of the same POI in an unaltered Gram positive bacterial cell.

2. The method of claim 1, wherein the altered Gram positive bacterial cell of step (b) comprises at least two genetic alterations that reduce expression of at least two genes selected from kinA, phrA and phrE.

3. The method of claim 1, wherein the altered Gram positive bacterial cell of step (b) comprises a genetic alteration that reduces expression of kinA, a genetic alteration that reduces expression of phrA and a genetic alteration that reduces expression of phrE.

4. The method of claim 1, wherein the genetic alteration is further defined as a decrease in the level of a kinA mRNA transcript, a decrease in the level of a phrA mRNA transcript and/or a decrease in the level of a phrEmRNA transcript.

5. The method of claim 1, wherein an increase in the expression of the POI is further defined as an increase in the level of the POI mRNA transcript.

6. The method of claim 1, wherein the altered Gram positive bacterial cell and the unaltered Gram positive bacterial cell further comprise at least one defective or inactive sporulation gene.

7. The method of claim 6, wherein the at least one defective or inactive sporulation gene is selected from the group consisting of Spo0A, SigF, SigG, SigE, SigK, spoIIAA, spoIIAB, spoIIR, spoIIGA, spoIIIAA, spoIIIAB, spoIIIAC, spoIIIAD, spoIIIAE, spoIIIAF, spoIIIAG, spoIIIAH, spoIVB, bofC, spoIVFA, spoIVFB, spoIVCA, spoIVCB, spoIIIC, and spoIIE.

8. The method of claim 1, wherein said altered Gram positive bacterial cell is a member of the Bacillusgenus

9. The method of claim 8, wherein the Bacillus cell is selected from the group consisting of B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii, and B. thuringiensis.

10. The method of claim 9, wherein the Bacillus cell is B. subtilis or B. licheniformis.

11. The method of claim 1, wherein said genetic alteration is a deletion of all or part of one or more of the kinA, phrA, and phrE genes.

12. The method of claim 1, wherein said genetic alteration results in a decrease in the activity of one or more of the KinA, PhrA, and PhrE proteins.

13. The method ofclaim 1, wherein the POI is an enzyme.

14. The method of claim 13, wherein the enzyme is selected from the group consisting of acetyl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, rhamno-galacturonases, ribonucleases, transferases, transport proteins, transglutaminases, xylanases, hexose oxidases, and combinations thereof.

15. The method ofclaim 13, wherein enzyme is a protease.

16. The method ofclaim 1, further comprising recoveringthe POI.

17. The method of claim 1, wherein the increased amount of an expressed POI relative to the unaltered Gram positive cell is at least 10% increased.

18. An altered Gram positive bacterial cell expressing an increased amount of a POI relative to the expression of the same POI in an unaltered Gram positive bacterial cell, wherein the altered bacterial cell comprises at least one genetic alteration that reduces expression of a kinA gene, a phrA gene or a phrEgene.

19. The altered cell of claim 18, wherein the altered cell comprises at least two genetic alterations that reduce expression of at least two genes selected from kinA, phrA and phrE.

20. The altered cell of claim 18, wherein the altered cell comprises a genetic alteration that reduces expression of kinA, a genetic alteration that reduces expression of phrA and a genetic alteration that reduces expression of phrE.

21. The altered cell of claim 18, wherein the altered cell and the unaltered cell further comprise at least one defective or inactive sporulation gene.

22. The cells of claim 21, wherein the at least one defective or inactive sporulation gene is selected from the group consisting of Spo0A, SigF, SigG, SigE, SigK, spoIIAA, spoIIAB, spoIIR, spoIIGA, spoIIIAA, spoIIIAB, spoIIIAC, spoIIIAD, spoIIIAE, spoIIIAF, spoIIIAG, spoIIIAH, spoIVB, bofC, spolVFA, spoIVFB, spoIVCA, spoIVCB, spoIIIC, and spoIIE.

23. The altered cell of claim 18, wherein the altered cell is a member of the Bacillus genus.

24. The altered cell of claim 23, wherein the Bacillus cell is selected from the group consisting of B. subtilis, B. licheniformis, B. lentus, B. brevis, B. stearothermophilus, B. alkalophilus, B. amyloliquefaciens, B. clausii, B. sonorensis, B. halodurans, B. pumilus, B. lautus, B. pabuli, B. cereus, B. agaradhaerens, B akibai, B. clarkii, B. pseudofirmus, B. lehensis, B. megaterium, B. coagulans, B. circulans, B. gibsonii, and B. thuringiensis.

25. The altered cell of claim 24, wherein the Bacillus cell is B. subtilis or B. licheniformis.

26. The altered cell of claim 18, wherein said genetic alteration is further defined as a decrease in the level of a kinA mRNA transcript, a decrease in the level of a phrA mRNA transcript and/or a decrease in the level of a phrE mRNA transcript.

27. The altered cell of claim 18, wherein said genetic alteration that reduces expression of a kinA gene, a phrA gene or a phrE gene is a deletion of all or part of the kinA, phrA, and phrE genes.

28. The altered cell of claim 18, wherein said genetic alteration results in a decrease in the activity the KinA, PhrA, and/or PhrE proteins.

29. The altered cell of claim 18, wherein the POI is an enzyme.

30. The altered cell of claim 29, wherein the enzyme is selected from the group consisting of acetyl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, rhamno-galacturonases, ribonucleases, transferases, transport proteins, transglutaminases, xylanases, hexose oxidases, and combinations thereof.

31. The altered cell of claim 29, wherein enzyme is a protease.

32. The altered cell of claim 18, further comprising recovering the POI.

33. The altered cell of claim 18, wherein the increased amount of an expressed POI relative to the unaltered Gram positive cell is at least 10% increased.
